SEQUENCE LISTING (54 sequences)

SEQ ID NO: 1 - protein, 25 aa, synthetic construct
AEMKTYSHRTMPSACTLVMCSSVES

SEQ ID NO: 2 - protein, 198 aa, synthetic construct
AEMKTYSHRTMPSACTLVMCSSVESDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 234 aa, synthetic construct
AEMKTYSHRTMPSACTLVMCSSVESGQAGPVGPKGDNGSVGEPGPKGDTGPSGPPGPPGVPDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - protein, 234 aa, synthetic construct
AEMKTYSHRTMPSACTLVMCSSVESGPLGKQGNIGPQGKPGPKGEAGPKGEVGAPGMQGSADVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 216 aa, synthetic construct
AEMKTYSHRTMPSACTLVMCSSVESGQAGPVGPKGDNGSVGEPDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 6 - protein, 216 aa, synthetic construct
AEMKTYSHRTMPSACTLVMCSSVESGPKGDTGPSGPPGPPGVPDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 7 - protein, 255 aa, synthetic construct
AEMKTYSHRTMPSACTLVMCSSVESGQAGPVGPKGDNGSVGEPGPKGDTGPSGPPGPPGVPGPAGREGPLGKQGNIGPQGKPDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 8 - protein, 288 aa, synthetic construct
AEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 9 - protein, 266 aa, synthetic construct
AEMKTYSHRTMPSACTLVMCSSVESGQAGPVGPKGDNGSVGEPGPKGDTGPSGPPGPPGVPDVASLRQQVEALQGQVQHLQAAFSQYKKVEDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 210 aa, synthetic construct
AEMKTYSHRTMPSACTLVMCSSVESDVASLRQQVEALQGQVQHLQAAFSQYKKVEGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 246 aa, synthetic construct
AEMKTYSHRTMPSACTLVMCSSVESGQAGPVGPKGDNGSVGEPGPKGDTGPSGPPGPPGVPDVASLRQQVEALQGQVQHLQAAFSQYKKVEGGGSGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 12 - DNA, 681 bp, synthetic construct
  source 1..681: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaaagcgacgtggccagcctgagacagcaggttgaagctctgcagggccaagtgcagcatctgcaggccgcctttagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 13 - DNA, 789 bp, synthetic construct
  source 1..789: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggacaggctggacctgtgggacccaagggcgataatggatctgtgggagagcctggacctaaaggcgatacaggcccttctggacctcctggaccacctggtgttccagatgtggccagcctgagacagcaggttgaagccctgcaaggccaggtgcagcatctgcaggctgccttcagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 14 - DNA, 789 bp, synthetic construct
  source 1..789: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggccctctgggcaagcagggcaatatcggacctcagggaaagcccggacctaagggcgaagctggaccaaaaggcgaagtgggagcacctggcatgcagggatctgctgatgtggccagcctgagacagcaggttgaagccctgcaaggccaggtgcagcatctgcaggctgccttcagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 15 - DNA, 735 bp, synthetic construct
  source 1..735: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggacaggctggacctgtgggacccaagggcgataatggatctgtgggcgaacctgatgtggccagcctgagacagcaggttgaagccctgcaaggccaggtgcagcatctgcaggctgccttcagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 16 - DNA, 735 bp, synthetic construct
  source 1..735: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggccccaagggcgatacaggaccttctggaccacctggacctccaggcgttccagatgtggccagcctgagacagcaggttgaagccctgcaaggccaggtgcagcatctgcaggctgccttcagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 17 - DNA, 852 bp, synthetic construct
  source 1..852: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggacaggctggacctgtgggacccaagggcgataatggatctgtgggagagcctggacctaaaggcgatacaggcccttctggacctcctggaccacctggcgttccaggacctgctggaagagaaggacctctgggcaagcagggcaatatcggccctcagggaaaacccgatgtggccagcctgagacagcaggttgaagccctgcaaggccaggtgcagcatctgcaggctgccttcagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 18 - DNA, 951 bp, synthetic construct
  source 1..951: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggcctgcctggcagagatggacgcgacggaagagaaggacctagaggcgagaaaggcgatcctggacttcctggtgctgctggacaagccggaatgccaggacaagctggacctgtgggacccaagggcgataatggatctgtgggagagcctgggcctaaaggcgatacaggaccttctggaccacctggacctccaggcgttccaggtcctgctggtagagagggacctctgggcaagcagggcaatatcggacctcagggaaagcccgatgtggccagcctgagacagcaggttgaagccctgcaaggccaggtgcagcatctgcaggctgccttcagccagtacaagaaggtggaaaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 19 - DNA, 885 bp, synthetic construct
  source 1..885: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggacaggctggacctgtgggacccaagggcgataatggatctgtgggagagcctggacctaaaggcgatacaggcccttctggacctcctggaccacctggtgttccagatgtggccagcctgagacagcaggttgaagccctgcaaggccaggtgcagcatctgcaggctgccttcagccagtacaagaaggtggaagatcctgccggcctgctggatctgagacagggaatgtttgcccagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatcaggatctgtgtctctggccctgcacctccagcctctgagatctgctgctggtgctgctgctctggctctgacagttgatctgcctcctgccagcagcgaggccagaaatagcgcctttggcttccaaggcagactgctgcatctgtctgccggacagagactgggagtgcatctgcacacagaagccagagctagacacgcctggcagctgacacaaggcgctacagtgctgggcctgttcagagtgacacctgagattccagccggactgccctctccaagaagcgagggcagcgactacaaggacgacgacgacaag

SEQ ID NO: 20 - DNA, 825 bp, synthetic construct
  source 1..825: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaatctggacaggctggacctgtgggacccaagggcgataatggatctgtgggagagcctggacctaaaggcgatacaggcccttctggacctcctggaccacctggtgttccagatgtggccagcctgagacagcaggttgaagccctgcaaggccaggtgcagcatctgcaggctgccttcagccagtacaagaaggtggaaggcggaggctctggcggtggaagtggcggaggatctaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 21 - DNA, 717 bp, synthetic construct
  source 1..717: mol_type = other DNA
atgggctggtcctgcatcatcctgtttctggtggccacagccacaggcgtgcactctgccgagatgaagacctacagccaccggaccatgcctagcgcctgtacactggtcatgtgcagcagcgtggaaagcgacgtggccagcctgagacagcaggttgaagctctgcagggccaagtgcagcatctgcaggccgcctttagccagtacaagaaggtggaaggcggcggatctggcggaggaagcggcggaggatctaaccctcagatcgccgctcacgtgatcagcgaggccagcagcaagacaacaagcgtgctgcagtgggccgagaagggctactacaccatgagcaacaacctcgtgaccctggaaaacggcaagcagctgaccgtgaagagacagggcctgtactacatctacgcccaagtgaccttctgcagcaacagagaggccagctctcaggccccttttatcgccagcctgtgcctgaagtcccctggcagattcgagagaatcctgctgagagccgccaacacacacagcagcgccaaaccttgtggacagcagtctatccacctcggcggagtgtttgaactgcagcctggcgcctccgtgttcgtgaatgtgaccgatccttctcaggtgtcccacggcaccggctttacatcctttggcctgctgaagctgggcagcgactacaaggacgacgacgacaag

SEQ ID NO: 22 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 23 - protein, 30 aa, synthetic construct
DVASLRQQVEALQGQVQHLQAAFSQYKKVE

SEQ ID NO: 24 - protein, 143 aa, synthetic construct
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 25 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 26 - protein, 146 aa, synthetic construct
GDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 27 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 28 - protein, 175 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 29 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 30 - protein, 171 aa, synthetic construct
NTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 31 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 32 - protein, 18 aa, synthetic construct
GPKGDTGPSGPPGPPGVP

SEQ ID NO: 33 - protein, 18 aa, synthetic construct
GQAGPVGPKGDNGSVGEP

SEQ ID NO: 34 - protein, 36 aa, synthetic construct
GQAGPVGPKGDNGSVGEPGPKGDTGPSGPPGPPGVP

SEQ ID NO: 35 - protein, 36 aa, synthetic construct
GPLGKQGNIGPQGKPGPKGEAGPKGEVGAPGMQGSA

SEQ ID NO: 36 - protein, 57 aa, synthetic construct
GQAGPVGPKGDNGSVGEPGPKGDTGPSGPPGPPGVPGPAGREGPLGKQGNIGPQGKP

SEQ ID NO: 37 - protein, 90 aa, synthetic construct
GLPGRDGRDGREGPRGEKGDPGLPGAAGQAGMPGQAGPVGPKGDNGSVGEPGPKGDTGPSGPPGPPGVPGPAGREGPLGKQGNIGPQGKP

SEQ ID NO: 38 - protein, 12 aa, synthetic construct
GGGSGGGSGGGS

SEQ ID NO: 39 - protein, 20 aa, synthetic construct
MLLFLLSALVLLTQPLGYLE

SEQ ID NO: 40 - protein, 19 aa, synthetic construct
MGLGLQWVFFVALLKGVHC

SEQ ID NO: 41 - protein, 19 aa, synthetic construct
MGWSCIILFLVATATGVHS

SEQ ID NO: 42 - protein, 7 aa, synthetic construct
DYKDDDK

SEQ ID NO: 43 - protein, 8 aa, synthetic construct
GDYKDDDK

SEQ ID NO: 44 - protein, 10 aa, synthetic construct
GSDYKDDDDK

SEQ ID NO: 45 - protein, 20 aa, synthetic construct
HHHHHHDYKDDDDKLVPRGS

SEQ ID NO: 46 - protein, 9 aa, synthetic construct
QKLISEEDL

SEQ ID NO: 47 - protein, 9 aa, synthetic construct
YPYDVPDYA

SEQ ID NO: 48 - protein, 11 aa, synthetic construct
QPELAPEDPED

SEQ ID NO: 49 - protein, 11 aa, synthetic construct
YTDIEMNRLGK

SEQ ID NO: 50 - protein, 452 aa, synthetic construct
AEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 51 - protein, 146 aa, synthetic construct
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 52 - protein, 375 aa, synthetic construct
AEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVENPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 53 - protein, 176 aa, synthetic construct
SDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 54 - protein, 55 aa, synthetic construct
AEMKTYSHRTMPSACTLVMCSSVESDVASLRQQVEALQGQVQHLQAAFSQYKKVE

(19) United States
(12) Patent Application Publication
(10) Pub. No.: US 2026/0015405 A1
(43) Pub. Date: Jan. 15, 2026

(54) FUSION PROTEIN COMPRISING A SURFACTANT-PROTEIN-D AND A MEMBER OF THE TNFSF

(71) Applicant: Transgene, Illkirch Graffenstaden (FR)

(72) Inventor: Jean-Baptiste MARCHAND, Illkirch-Graffenstaden (FR)

(21) Appl. No.: 18/876,073

(22) PCT Filed: Jun. 30, 2023
(86) PCT No.: PCT/EP2023/068005

§ 371 Date: Dec. 17, 2024

(30) Foreign Application Priority Data
Jul. 1, 2022 (EP) 22305974.2

(51) Int. Cl. C07K 14/705; A61K 35/768; A61K 38/00; C07K 14/785; C12N 15/86

(52) U.S. Cl. CPC C07K 14/70575; A61K 35/768; C07K 14/785; C12N 15/86; A61K 38/00; C07K 2319/00; C12N 2710/24132; C12N 2710/24143; C12N 2710/24151

(57) ABSTRACT

The disclosure is in the field of immunology and oncology, especially for treating, preventing, or inhibiting proliferative diseases, particularly cancer, infectious diseases and disorders associated with dysfunction of TNF cytokines. The disclosure relates to a novel SPD-TNFSF fusion protein including a TNF-superfamily (TNFSF) ligand, or receptor binding domain thereof, fused to a coiled-coil domain of surfactant protein-D (SPD). Also provided a trimeric or multimeric fusion protein including a plurality of SPD-TNFSF fusion proteins. The disclosure also provides an expression vector as mRNA, plasmid or virus including an isolated nucleotide sequence encoding the SPD-TNFSF fusion protein and a cell or a pharmaceutical composition including thereof.

### TECHNICAL FIELD OF THE INVENTION

The present invention generally relates to the field of immunology and oncology and more specifically to compositions and methods to treat, prevent, or inhibit proliferative diseases, particularly cancer, infectious diseases and disorders associated with dysfunction of TNF cytokines.

Embodiments include a SPD-TNFSF fusion protein comprising a TNF-superfamily (TNFSF) ligand, or receptor binding domain thereof, fused to a coiled-coil domain of surfactant protein-D (SPD), a trimeric or multimeric fusion protein comprising a plurality of SPD-TNFSF fusion proteins. Embodiments also include expression vectors such as a mRNA, a plasmid and a virus comprising nucleotide sequence encoding one or more SPD-TNFSF fusion protein. The SPD-TNFSF fusion protein, the isolated nucleotide sequence, the mRNA, the plasmid and the virus encoding the SPD-TNFSF fusion protein are suitable for pharmaceutical composition and its use for treating cancer and infectious diseases, and more generally proliferative diseases and disorders associated with dysfunction of TNF cytokines.

### BACKGROUND ART

Cancer is caused by both external factors (e.g. tobacco, infectious organisms, alimentary habits, chemicals, and radiation) and internal factors (e.g. inherited mutations, hormones, immune conditions, and mutations that occur from metabolism). Each year, cancer is diagnosed in more than 12 million subjects worldwide. In industrialized countries, approximately one person out of five will die of cancer. Although a vast number of chemotherapeutics exist, they are often ineffective and there is still an unmet medical need for effective and less toxic therapies, especially for patients who are resistant to existing treatments.

An effective T-cell response requires functional and optimal innate and adaptative immunity, which depends in particular on multiple specific interactions between different cells through soluble stimulus and surface linker/receptor binding. In this context, the CD8+ cytotoxic T lymphocyte (CTL) responses play a crucial role in antitumor immunity (Ara et al., 2018, ImmunoTargets and therapy, 7, 55-61). Three signals have been mentioned as key parameter. The first signal concerns the binding of antigen-specific T-cell receptor (TCR) to peptide-loaded major histocompatibility complex (MHC) on antigen-presenting cells (APCs). The second signal is generated through the engagement of costimulatory molecules, namely B7-1 (CD80)/B7-2 (CD86) and CD28 (e.g., T-cell CD28/APC CD80), consequent to Ag-specific T cell-APC interactions. The third signal induced by the secretion of cytokines, enhances and modifies the responding effector CTLs.

Tumor necrosis factor (TNF) superfamily as costimulatory molecules fulfils a crucial role in the immune regulation and antitumor immunity (Vinay et al., 2009, Cell Biol Int.; 33(4):453-465). TNFSF members are well known for mediating immune response through T cells shaping properties. Previous investigations allow to identify the following TNFSF members: CD40L, 4-1-BBL, OX40L, CD70, TNF, GITRL, LIGHT, FASL, TWEAK, APRIL, RANKL, TRAIL, CD30L, NGF, Baff, LTβ, LTα, LTαβ2, TL1A, TLA, EDA.

Efficient antigen recognition by antigen-specific T cells critically depends on the presence and functionality of specialized antigen-presenting cells (APC), such as B cells and dendritic cells (DCs). For example, while CD40 is expressed on many cell types, including macrophages, B cells and DCs, its ligand CD40L is mostly expressed on activated CD4+ T cells. CD40L is expressed on surface of activated B, T and NK cells but also on adipose cells, and basophils (Richards et al. Hum Vaccin Immunother. 2020; 16(2):377-387). The direct interaction between CD40L expressed on CD4+ T cells and CD40 expressed on DCs, “licenses” DCs to prime CD8 T cell responses, by up-regulation of co-stimulatory molecules. Such CD40L-CD40 interaction leads to the activations of CD40 bearing cells which then express adhesion (ICAM), co-stimulatory (CD80/CD86), and presenting MHC I and II molecules in addition of cytokines/chemokines (TNFα, IL6 . . . ). In the tumor microenvironment, adhesion molecules and cytokine/chemokines act together to induce the infiltration and activation of immune cells, that ultimately destroy tumor cells and skew the tumor from an immunosuppressive to an immunocompetent microenvironment (Richards et al. Hum Vaccin Immunother. 2020; 16(2):377-387). Similarly, the TNFSF receptors 4-1-BB, OX40, GITR and CD27 are expressed on T cells and respond to costimulation by ligands expressed on APC, lymphocytes, and innate immune cells. These various studies have highlighted the interest of developing TNFSF ligands to treat proliferative diseases such as cancer and disorders associated with dysfunction of TNF cytokines such as infectious diseases.

In the recent years, the TNF superfamily ligands have emerged as attractive candidates for the development of vaccines and immunotherapies, more specially such as an alternative approach for the design of novel molecular adjuvants (Gupta et al., 2013, Immunol Res., 57(1-3):303-10). The adjuvantation can change strength, quality, and functionality of innate and adaptive immune responses with a focus on rapid induction of a large number of CD8 T cells able to protect against specific diseases (Lauterbach et al., 2013, Front Immunol., 27; 4:251). A recombinant MVA-CD40L has already demonstrated the potential of CD40L-adjuvanted virus to induce rapidly strong antigen-specific CD8T cell responses for the development of prophylactic and therapeutic vaccines against cancers and infectious diseases such as HIV/AIDS, Ebola and Marburg hemorrhagic fever, malaria and hepatitis C (Lauterbach et al., 2013, Front Immunol. 2013 Aug. 27; 4:251). An approach to arm an adenovirus with CD40L has also been developed to stimulate beneficial immunologic responses for the treatment of tumors (Pesonen et al., 2012, Cancer Res., 72(7)). In the same way, by providing a robust T-cell responses, a recombinant 4-1-BBL adenovirus is a promising adjuvant for human memory CD8 T cells and suggests being beneficial in antiviral therapeutic strategies (Bukczynski et al., 2004, Proc Natl Acad Sci USA.; 101(5):1291-1296).

It has also been recognized by the skilled person that TNFSF ligands generally need to be homo-multimerized to fully activate responding cells. TNFSF signalling, which is also structurally well-defined, requires appropriate receptor clustering and at least trimerization. While the TNFSF ligands exist as trimeric units by self-assembling, the receptors are usually separated on the cell surface (Richards et al. 2020, Hum Vaccin Immunother., 16(2):377-387). The interaction between multimeric TNFSF ligands and their corresponding receptors leads to a precise receptor clustering and is a prerequisite for producing signal transmission into the cells (Kucka et al., 2020, Front Cell Dev Biol., 8:615141). As previously shown, the higher order oligomeric TNFSF ligands, particularly the dodecameric TNFSF ligands are more effective in signalling than single trimeric TNFSF ligands (Haswell et al. 2001, Eur J Immunol., 31(10):3094-100).

Due to the critical role of TNFSF ligands in immune response, more specifically in tumoral response, various strategies have been explored to enhance their agonist properties, like the fusion with Surfactant-Protein D as collectin family members.

First recombinant fusion proteins comprising a TNF cytokine and a di- or multimerization domain to increase avidity of ligands have been disclosed by the patent application WO01/49866. However, the multimerization of the TNF protein was rather inefficient due to the molecular weight of the multimerization domain. WO01/42298 discloses the method for constructing stable bioactive fusion proteins to express TNFSF, more particularly CD40L and RANKLE/TRANCE members, with collectin, more particularly SPD comprising a signal sequence, a collagen domain, and a coiled-coil neck domain. The large size of the resulting fusion proteins makes them less likely to diffuse into the tumor, thereby limiting their potential activities. While this property may be especially useful to prevent them from clearance, these fusion proteins have limited CD40 agonist activity.

WO2009/007120 also discloses fusion protein comprising a TNFSF cytokine fused to a collectin trimerization domain. Although these fusion proteins may form multimeric proteins, there are restricted forming trimeric form by the inability to self-assemble more than three monomeric units, thus reducing their potential agonist activity.

The TNFSF receptors are ubiquitously expressed on normal cells, as a consequence of which TNFSF ligands have no or only limited binding selectivity and considerable toxicity. While TNFSF ligands axis remains of great interest in cancer immunotherapy, systemic TNFSF treatment is associated with severe dose-limiting toxicity and yields minimal clinical activity (Bremer et al., 2013, ISRN Oncol.; 2013:371854). In a first clinical study of a CD40 agonist, the majority of patients presented a cytokine release syndrome that produced fevers, rigors and chills (Vonderheide et al., 2007, J Clin Oncol., 25(7):876-883). Evidence of hepatotoxicity is also commonly observed with CD40 agonists (Beatty et al., 2017, Expert Rev Anticancer Ther.; 17(2):175-186). Thus, tumor-restricted activation and selective enhancement of TNFSF-mediated T-cells stimulation in the tumor environment are being continued.

### Technical Problem

One may expect that cancer will continue to be a serious global health threat for many years due to the high number of causal factors that may act together or separately to initiate or promote the development of a cancer. Moreover, malignant and especially metastatic tumors are often resistant to conventional therapies explaining the significant morbidity of some cancers.

Thus, there is an important need to develop more effective approaches, for improving prevention and treatment of cancer and more generally proliferative diseases and disorders associated with dysfunction of TNF cytokines. While it is known that multimeric complexes of TNFSF cytokines are difficult to prepare from recombinant monomeric forms, the inventors have generated SPD-TNFSF fusion protein, which provide efficient multimerization properties and enhance the agonistic properties of the ligand by optimal clustering of its receptor and independent of the cellular environment. The optimal formats have a relatively small molecular size allowing their diffusion into tumor and their efficient elimination by renal clearance. This relatively small size is also an advantage in case of insertion in vectors with limited capacity (some virus for example). Indeed, it is well known that tissue distribution, or cancer penetration, is better for a smaller therapeutic than for bigger ones (Li et al. MAbs. 2016; 8(1):113-9). A person stated in the art will well know the biodistribution of such a molecule is therefore enhanced.

Due to limiting binding selectivity and deleterious off-target toxicity from systemic TNFSF receptors activation, there is also a need to develop a novel targeting SPD-TNFSF fusion protein which improves T cell responses by the likely synergistic action of the viral replication and TNFSF stimulation on tumor cells and immune cells.

Other and further aspects, features and advantages of the present invention will be apparent from the following description of the presently preferred embodiments of the invention. These embodiments are given for the purpose of disclosure.

### DESCRIPTION OF THE FIGURES

FIGS. 1A and 1B show level of expression of SPD-TNFSF constructions (pTG19965, pTG19966, pTG19967, pTG19968, pTG19969) by infected/transfected HeLa cells. Clarified supernatants containing the different SPD-TNFSF molecules were loaded on SDS-PAGE under reducing and non-reducing conditions. Proteins were transferred on PDVF membrane and hybridized with HRP-conjugated anti-FLAG-tag for immunodetection. Blot A: different constructions of SPD-CD40L. Blot B: pTG19967 construct loaded at different volumes. Arrowheads indicate oligomers larger than trimer.

FIG. 2 shows CD40L agonist activity of culture medium of HeLa cells infected with vaccinia virus and then transfected with different plasmids carrying cassette encoding SPD-TNFSF constructions (pTG19965, pTG19966, pTG19967, pTG19968, pTG19969) and under control of pH5.R promoter (i.e. poxvirus promoter). HEK cells modified to express the reporter enzyme SEAP under the control of CD40 inducible promoter were incubated with different dilutions of the clarified supernatants containing the SPD-TNFSF constructions. SEAP enzymatic activity in culture medium was measured after 20-24 hours incubation. Negative controls were non-infected cells (noted medium) or cells infected and transfected with a plasmid without transgene (pTG19333) or with a plasmid encoding an irrelevant FLAG-tagged protein (pTG19274). Results are reported as the SEAP activity versus 1/dilution of the culture medium and as the means and standard deviations of two measurements.

FIG. 3 shows CD40 binding activity of culture medium of HeLa cells infected with vaccinia virus and then transfected with different plasmids carrying cassette encoding SPD-TNFSF constructions (pTG19325, pTG19344, pTG19965, pTG19966, pTG19967, pTG19968, pTG19969) and under control of pH5.R promoter (i.e. poxvirus promoter). CD40-Fc was coated on 96-well plate and different dilutions of the clarified supernatants containing the TNFSF constructions were applied. The bound specific protein was detected using a non-blocking anti-CD40L monoclonal antibody followed by a HRP-conjugated anti-IgG. Negative control was cells infected and transfected with a plasmid encoding an irrelevant FLAG-tagged protein (pTG19274). Results are reported as the OD 450 nm versus 1/dilution of the culture medium and as the mean and standard deviation of two measurements or as single measurement.

FIG. 4 shows CD40 binding activity of culture medium of HeLa cells infected with vaccinia virus and then transfected with different plasmids carrying cassette encoding SPD-TNFSF constructions (pTG19965, pTG19966, pTG19968, pTG20038 to pTG20042) and under control of pH5.R promoter (i.e. poxvirus promoter). CD40-Fc was coated on 96-well plate and different dilutions of the clarified supernatants containing the TNFSF constructions were applied. The bound specific protein was detected using a non-blocking anti-CD40L monoclonal antibody followed by a HRP-conjugated anti-IgG. Negative control was cells infected without transfection (VVTG18058). Results are reported as OD 450 nm versus 1/dilution of the culture medium.

FIG. 5 shows CD40L agonist activity of culture medium of HeLa cells infected with vaccinia virus and then transfected with different plasmids carrying cassette encoding SPD-TNFSF constructions (pTG19965, pTG19966, pTG19968, pTG20038 to pTG20042) and under control of pH5.R promoter (i.e. poxvirus promoter). HEK cells modified to express the reporter enzyme SEAP under the control of CD40 inducible promoter were incubated with different dilutions of the clarified supernatants containing the SPD-TNFSF constructions. SEAP enzymatic activity in culture medium was measured after 20-24 hours incubation. Negative controls were cells infected without transfection (VVTG18058) or non-infected cells (noted medium). Results are reported as the SEAP activity versus 1/dilution of the culture medium.

FIG. 6 shows CD40 binding activity of culture medium of HeLa cells infected by COPTG19967, COPTG19968, and COPTG19969 measured as described in FIG. 4. Negative control was cells infected with VVTG18058. Results are reported as OD 450 nm versus 1/dilution of the culture medium.

FIG. 7 shows CD40 agonist activity of culture medium of HeLa cells infected by COPTG19967, COPTG19968, and COPTG19969 measured as described in FIG. 3. Negative control were cells infected with VVTG18058 or culture medium of non-infected cells (noted medium). Results are reported as the SEAP activity versus 1/dilution of the culture medium.

FIG. 8 shows level of expression of 4-1BBL constructions (pTG20032 encoding 4-1BBL ectodomain alone and pTG20033 encoding a SPD-4-1BBL fusion protein according to the present invention) by infected/transfected HeLa cells. Clarified supernatants containing the different constructs were loaded on SDS-PAGE under reducing and non-reducing conditions. Proteins were transferred on PDVF membrane and hybridized with HRP-conjugated anti-FLAG-tag monoclonal antibody for immunodetection. Negative control was cells infected with a plasmid encoding an irrelevant FLAG-tagged protein (pTG19274).

FIG. 9 shows 4-1BB agonist activity of culture medium of HeLa cells infected/transfected with 4-1BBL constructions (pTG20032 encoding 4-1BBL ectodomain alone and pTG20033 encoding a SPD-4-1BBL fusion protein according to the present invention) by using Promega 4-1BB reporter cells. Negative control was cells infected with a plasmid encoding an irrelevant FLAG-tagged protein (pTG19274).

### SUMMARY OF THE INVENTION

The present invention concerns a SPD-TNFSF fusion protein comprising or consisting of a N-terminus domain, a coiled-coil neck domain of surfactant protein-D (SPD) between the N-terminus domain and the C-terminus position, and a TNF-superfamily (TNFSF) ligand, or a receptor binding domain thereof in C-terminus position.

In one embodiment, the SPD-TNFSF fusion protein comprises or consists of a N-terminus domain, a coiled-coil neck domain of surfactant protein-D (SPD) between the N-terminus domain and the C-terminus position, and a TNF-superfamily (TNFSF) ligand, or a receptor binding domain thereof in C-terminus position, wherein the N-terminus domain and the coiled-coil neck domain of SPD are directly fused without amino acid residues in between. In one embodiment, the N-terminus domain and the coiled-coil neck domain of SPD in said SPD-TNFSF fusion protein consists of a sequence of SEQ ID NO:54.

In one embodiment, the SPD-TNFSF fusion protein further comprises a collagen domain between the N-terminus domain and the coiled-coil neck domain of SPD. The said collagen domain comprises or consists of between 1 and 40 (GXX) repeats, preferably between 3 and 30 (GXX) repeats, preferably between 6 and 20 (GXX) repeats, more preferably 12 (GXX) repeats, wherein X is an amino acid, and G is a glycine amino acid.

X may be identical or different in each repeat, for example but not limited to, a repeat may be GAA or a repeat may be GTA.

Alternatively or in combination, the SPD-TNFSF fusion protein further comprises a linker between the coiled-coil neck domain and the TNF-superfamily ligand or the receptor binding domain thereof. The said linker is a glycine/serine linker and has a length of 4-20 amino acids, preferably 8-16, more preferably 12 amino acids.

The TNFSF ligand is preferably selected from CD40L, 4-1-BBL, OX40L, CD70, TNF, GITRL, LIGHT, FASL, TWEAK, APRIL, RANKL, TRAIL, CD30L, NGF, Baff, LTβ, LTα, LTαβ2, TL1A, TLA, EDA, more preferably CD40L or 4-1-BBL.

The N-terminus domain has at least 85%, preferably at least 90%, and more preferably at least 95%, or has 100% identity with the amino acid sequence shown in SEQ ID NO:1.

In one embodiment, the SPD-TNFSF fusion protein comprises or consists of a sequence selected from SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11.

The present invention further concerns a trimeric fusion protein comprising three SPD-TNFSF fusion proteins. The present invention also concerns a multimeric fusion protein comprising a plurality of trimeric fusion proteins and forming a hexamer, a dodecamer, an octadecamer or a highly-order oligomer, preferably a hexamer, and more preferably a dodecamer.

The present invention also concerns an isolated nucleotide sequence encoding a SPD-TNFSF fusion protein. In one embodiment, the isolated nucleotide sequence comprises or consists of a sequence selected from SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20 or SEQ ID NO:21.

The present invention further relates to an expression vector such as mRNA, plasmid or virus comprising an isolated nucleotide sequence encoding a SPD-TNFSF fusion protein. In one embodiment, the said virus is an oncolytic virus selected from the group consisting of poxvirus, herpes virus, reovirus, Seneca Valley virus (SVV), vesicular stomatitis virus (VSV), Newcastle disease virus (NDV), morbillivirus, retrovirus, adenovirus, adenovirus-associated virus (AAV), herpes simplex virus (HSV), measles virus, foamy virus, alpha virus, lentivirus, influenza virus, Sindbis virus, rhabdovirus, picornavirus, coxsackievirus, parvovirus or chimeras thereof, with a specific preference for poxvirus. In a further embodiment, the said poxvirus belongs to the Orthopoxvirus genus, preferably selected from the group consisting of Vaccinia virus, cowpox virus, canarypox virus and ectromelia virus or chimeras thereof, with a specific preference for Vaccinia virus and in particular a vaccinia virus selected from the group of Elstree, Wyeth, Copenhagen, Lister, Tian Tian and Western Reserve strains. In another embodiment, the said poxvirus belongs to leporipoxvirus genus selected from the group consisting of myxoma virus, rabbit fibroma virus and squirrel fibroma virus with a specific preference for the myxoma virus. In a preferred embodiment, the oncolytic poxvirus is a modified poxvirus defective for thymidine kinase (TK) activity resulting from inactivating mutations in the J2R viral gene. Alternatively or in combination, the oncolytic poxvirus is defective for ribonucleotide reductase (RR) activity resulting from inactivating mutations in the viral I4L and/or F4L gene(s).

Alternatively to or in combination with, the modified poxvirus may be further modified, in the M2L locus (preference for modification leading to a suppressed expression of the viral m2 protein), resulting in a modified poxvirus defective m2 functions (m2-defective poxvirus).

In another embodiment, the said virus is a non-oncolytic virus, preferably a poxvirus, selected from the group consisting of Pseudocowpox virus (PCPV), Modified vaccinia Virus Ankara (MVA), highly attenuated vaccinia virus strain (NYVAC), Swinepox virus (SWPV), Fowlpox virus (FPV) or chimeras thereof.

The present invention further concerns a method for producing the said virus comprising the steps of a) preparing a producer cell b) transfecting or infecting the prepared producer cell with the virus, c) culturing the transfected or infected producer cell under suitable conditions so as to allow the production of the virus, d) recovering the produced virus from the culture of said produced cells and optionally e) purifying said recovered virus.

The present invention further concerns a cell comprising the nucleotide sequence, the mRNA, the plasmid or the virus of the invention.

The present invention also concerns the SPD-TNFSF fusion protein, the trimeric fusion protein, the multimeric fusion protein, the nucleotide sequence, the mRNA, the plasmid, the virus or the cell for the use in medicine, preferably in the treatment of proliferative diseases such as cancer and disorders associated with dysfunction of TNF cytokines such as infectious diseases, inflammatory diseases, metabolic diseases, autoimmune diseases, degenerative diseases, apoptosis-associated diseases and transplant rejections, more preferably proliferative diseases and even more preferably in the treatment of cancer. The present invention further concerns the SPD-TNFSF fusion protein, the trimeric fusion protein, the multimeric fusion protein, the nucleotide sequence, the mRNA, the plasmid, the virus or the cell in combination with one or more chemotherapeutic drugs or immunotherapeutic products effective for use in the treatment of cancer.

The present invention further provides a pharmaceutical composition comprising or consisting of the SPD-TNFSF fusion protein, a nucleotide sequence, a mRNA, a plasmid, a virus or a cell of the invention and optionally a pharmaceutically acceptable diluent, carrier, vehicle and/or excipient. In one embodiment, the pharmaceutical composition further comprises one or more effective chemotherapeutic drugs or immunotherapeutic products. In one embodiment, the pharmaceutical composition is used for the treatment of cancer. In one embodiment, the pharmaceutical composition for use is administered via parenteral route, more preferably via intravenous, subcutaneous, or intramuscular route, and even more preferably via intravenous route.

The present invention also concerns a method of treatment of cancer in a subject comprising administering a therapeutically effective amount of the SPD-TNFSF fusion protein, the nucleotide, the mRNA, the plasmid, the virus, the cell or the pharmaceutical composition of the invention.

### DETAILED DESCRIPTION OF THE INVENTION

The present invention relates to a SPD-TNFSF fusion protein and a virus comprising inserted in its genome one or more molecule(s) encoding one or more SPD-TNFSF fusion protein(s).

### Definitions

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “one or more” refers to either one or a number above one (e.g. 2, 3, 4, 5, etc.).

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues. “Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues. “Consisting of” means excluding more than trace elements of other components or steps. For example, a polypeptide “consists of” an amino acid sequence when the polypeptide does not contain any amino acids but the recited amino acid sequence.

The terms “polypeptide”, “peptide” and “protein” refer to polymers of amino acid residues which comprise at least nine or more amino acids bonded via peptide bonds. The polymer can be linear, branched or cyclic and may comprise naturally occurring and/or amino acid analogs and it may be interrupted by non-amino acids. As a general indication, if the amino acid polymer is more than 50 amino acid residues, it is preferably referred to as a “polypeptide” or a “protein” whereas if it is 50 amino acids long or less, it is referred to as a “peptide”.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (RNA) (e.g. mRNA, antisense RNA, siRNA) or mixed polyribo-polydeoxyribonucleotides. They encompass single or double-stranded, linear or circular, natural or synthetic, modified or unmodified polynucleotides. Moreover, a polynucleotide may comprise non-naturally occurring nucleotides and may be interrupted by non-nucleotide components.

The term “analog” or “variant” as used herein refers to a molecule (polypeptide or nucleic acid) exhibiting one or more modification(s) with respect to the native counterpart. Any modification(s) can be envisaged, including substitution, insertion and/or deletion of one or more nucleotide/amino acid residue(s). Preferred are analogs that retain a degree of sequence identity of at least 80%, preferably at least 85%, more preferably at least 90%, more preferably at least 95% and even more preferably at least 98% identity with the sequence of the native counterpart.

In a general manner, the term “identity” refers to an amino acid to amino acid or nucleotide to nucleotide correspondence between two polypeptide or nucleic acid sequences. The percentage of identity between two sequences is a function of the number of identical positions shared by the sequences, taking into account the number of gaps which need to be introduced for optimal alignment and the length of each gap. Various computer programs and mathematical algorithms are available in the art to determine the percentage of identity between amino acid sequences, such as for example the Blast program available at NCBI or ALIGN in Atlas of Protein Sequence and Structure (Dayhoffed, 1981, Suppl., 3: 482-9). Programs for determining identity between nucleotide sequences are also available in specialized data base (e.g. Genbank, the Wisconsin Sequence Analysis Package, BESTFIT, FASTA and GAP programs). For illustrative purposes, “at least 80% identity” means 80%, 81%, 82%, 83%, 84%, 85%, 86%, 87%, 88%, 89%, 90%, 91%, 92%, 93%, 94%, 95%, 96%, 97%, 98%, 99% or 100%.

As used herein, the term “isolated” refers to a protein, polypeptide, peptide, polynucleotide, vector, etc., that is removed from its natural environment (i.e. separated from at least one other component(s) with which it is naturally associated or found in nature). For example, a nucleotide sequence is isolated when it is separated of sequences normally associated with it in nature (e.g. dissociated from a genome) but it can be associated with heterologous sequences.

The term “obtained from”, “originating” or “originate” is used to identify the original source of a component (e.g. polypeptide, nucleic acid molecule) but is not meant to limit the method by which the component is made which can be, for example, by chemical synthesis or recombinant means.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. In the context of the invention, the term “host cells” include prokaryotic cells, lower eukaryotic cells such as yeast, and other eukaryotic cells such as insect cells, plant and mammalian (e.g. human or non-human) cells as well as cells capable of producing the virus (oncolytic or non-oncolytic virus) and/or the fusion protein for use in the invention. This term also includes cells which can be or has been the recipient of the vectors described herein as well as progeny of such cells.

The terms “virus”, “viral particle”, “viral vector” and virion” are used interchangeably and are to be understood broadly as meaning a vehicle comprising at least one element of a wild-type virus genome and may be packaged into a viral particle or to a viral particle. Although, viral particles may or may not contain nucleic acid (i.e. the viral genome) it is preferred that a virus comprises a DNA or RNA viral genome packaged into a viral particle (or virion) and is infectious (i.e. capable of infecting and entering a host cell or subject). Desirably, the virus of this invention comprises a DNA genome, and most preferably a double-stranded DNA genome. In the context of the present disclosure, a “virus” includes wild-type and engineered (modified) viruses. Modification(s) can be within endogenous viral genes (e.g. coding and/or regulatory sequences) and/or within intergenic regions. Moreover, modification(s) can be silent or not (e.g. resulting in a modified viral gene product). Modification(s) can be made in a number of ways known to those skilled in the art using conventional molecular biology techniques. Desirably, the modifications encompassed by the present invention affect, for example virulence, toxicity, pathogenicity, or replication of the virus compared to a virus without such modification, but do not completely impair infection and production at least permissive cells.

The term “oncolytic virus” encompasses any virus naturally occurring, engineered or otherwise modified. As used herein, the term “oncolytic virus” refers to a virus capable of selectively replicating in dividing cells (e.g. a proliferative cell such as a cancer cell) with the aim of slowing the growth and/or lysing said dividing cell, either in vitro or in vivo, while showing no or minimal replication in non-dividing cells (e.g. primary cells). Typically, an oncolytic virus contains a viral genome packaged into a viral particle (or virion) and is infectious (i.e. capable of infecting and entering into a host cell or subject). As used herein, this term encompasses DNA or RNA vector (depending on the virus in question) as well as viral particles generated thereof.

As used therein, the terms “chimeras”, “virus chimeras” or “chimeras of viruses” refers to viruses obtained by homologous recombination between several distinct strains of viruses. Several chimeras obtained by mixing genomes from different poxviruses have been described and are available to the skilled person (such as CF189 chimeras obtained from ORF and pseudocowpox viruses (Choi et al, Novel chimeric parapoxvirus CF189 as an oncolytic immunotherapy in triple-negative breast cancer. Surgery Volume 163, Issue 2, February 2018, Pages 336-342); CF33 chimera obtained from multiple strains of VV, cowpox, and rabbitpox (Chaurasiya, S. et al., 2020, Cancer Gene Ther 27, 125-135).

The term “non-oncolytic virus” encompasses any virus which is not defined as an oncolytic virus.

The term “treatment” (and any form of treatment such as “treating”, “treat”) as used herein encompasses prophylaxis (e.g. preventive measure in a subject at risk of having the pathological condition to be treated) and/or therapy (e.g. in a subject diagnosed as having the pathological condition), eventually in association with conventional therapeutic modalities. The result of the treatment is to slow down, cure, ameliorate or control the progression of the targeted pathological condition. For example, a subject is successfully treated for a cancer if after administration of a fusion protein as described herein, a virus as described herein or a combination thereof as described herein, the subject shows an observable improvement of its clinical status.

The term “administering” (or any form of administration such as “administered”) as used herein refers to the delivery to a subject of a therapeutic agent such as the virus described herein

The term “proliferative disease” as used herein encompasses any disease or condition resulting from uncontrolled cell growth and spread uncontrolled cell growth and spread including cancer and some cardiovascular diseases (restenosis that results from the proliferation of the smooth muscle cells of the blood vessel wall, etc.). The term “cancer” may be used interchangeably with any of the terms “tumor”, “malignancy”, “neoplasm”, etc. The terms are meant to include any type of tissue, organ or cell, any stage of malignancy (e.g. from a prelesion to stage IV).

The term “disorders associated with TNF cytokine dysfunction” as used herein encompasses any disease, disorder or condition resulting in TNF signalling dysfunction.

The term “subject” generally refers to an organism for whom any product and method of the invention is needed or may be beneficial. Typically, the organism is a mammal, particularly a mammal selected from the group consisting of domestic animals, farm animals, sport animals, and primates. Preferably, the subject is a human who has been diagnosed as having or at risk of having a proliferative disease such as a cancer or disorders associated with TNF cytokine dysfunction. The terms “subject” and “patients” may be used interchangeably when referring to a human organism and encompasses male and female. The subject to be treated may be a new-born, an infant, a young adult, an adult or an elderly.

The term “combination” or “association” as used herein refers to any arrangement possible of various components (for example a fusion protein or a virus and one or more substance effective in anticancer therapy). Such an arrangement includes mixture of said components as well as separate combinations for concomitant or sequential administrations. The present invention encompasses combinations comprising equal molar concentrations of each component as well as combinations with very different concentrations. It is appreciated that optimal concentration of each component of the combination can be determined by the artisan skilled in the art.

The terms “chemotherapeutic drugs” or “immunotherapeutic products” as used herein refer to a product comprising one or more antigen(s) which is expected to induce or activate an immune response, whether specific or non-specific, humoral, or cellular, when delivered appropriately to a subject.

### SPD-TNFSF Fusion Protein

As used herein, the term “fusion protein” refers to a covalent linkage in a single protein chain of two or more polypeptides which is performed by genetic means i.e. by fusing in frame the nucleic acid molecules encoding each of said polypeptides or fragment thereof. By “fused in frame”, it is meant that the expression of the fused coding sequence results in a single polypeptide with functional properties derived from each of the original polypeptides. The fusion can be direct (i.e. without additional amino acid residues in between) or indirect (e.g. through a linker between the fused polypeptides).

The term “SPD” or “surfactant-protein D” as used herein refers to a functional fragment of the native surfactant-protein D or derivatives thereof. The SPD is preferably of mammalian origin such as human, mouse, rabbit, non-human primates or pig origin, preferably of human or non-human primates origin, more preferably of human origin.

In the context of the present invention, the SPD-TNFSF fusion protein comprises or consists of the fusion of a surfactant-protein D (SPD) and a TNFSF ligand, or a receptor binding domain thereof. In the present invention, SPD comprises or consists of a N-terminus domain and a coiled-coil neck domain. In one embodiment, the N-terminus domain and the coiled-coil neck domain are directly fused without amino acid residues in between. In one embodiment, the N-terminus domain and the coiled-coil neck domain of SPD in said SPD-TNFSF fusion protein consists of a sequence of SEQ ID NO:54.

The N-terminus domain of the present invention comprises an amino acid sequence having at least 85%, preferably at least 90% and more preferably at least 95%, or has 100% identity with the amino acid sequence shown in SEQ ID NO:1 particularly amino acids 21-45 of native human SPD (SEQ ID NO:22).

The coiled-coil neck domain of the present invention comprises an amino acid sequence having at least 85%, preferably at least 90% and more preferably at least 95% identity with the amino acid sequence shown in SEQ ID NO:23, particularly amino acids 223-252 of native human SPD (SEQ ID NO:22).

The TNFSF ligand may be selected from the TNF superfamily members including without limitation CD40L, 4-1-BBL, OX40L, CD70, TNF, GITRL, LIGHT, FASL, TWEAK, APRIL, RANKL, TRAIL, CD30L, NGF, Baff, LTβ, LTα, LTαβ2, TL1A, TLA, EDA or a receptor binding thereof. The TNFSF ligand is preferably of mammalian origin such as human, mouse, rabbit, non-human primates or pig origin, preferably of human or non-human primates origin, more preferably of human origin.

In preferred embodiments, the member of the TNFSF or receptor binding domain thereof is selected from:
CD40L, 4-1BBL, Baff, APRIL, EDA, GITRL, OX40L, CD70, TL1A, LIGHT, LTαβ₂, RANKL, TWEAK, FASL, TRAIL, TNF and LTa or receptor binding domain thereof;
Category II TNFSF members, preferably from CD40L, 4-1BBL, Baff, APRIL, EDA, OX40L, CD70, TWEAK, FASL, TRAIL, and TNF or receptor binding domain thereof;
TNFSF members involved in immune cell activation, preferably from CD40L, 4-1BBL, GITRL, OX40L, CD70, TL1A or receptor binding domain thereof;
category II TNFSF members involved in immune cell activation, preferably from CD40L, 4-1BBL, OX40L, CD70 or receptor binding domain thereof; or
CD40L, 4-1BBL or receptor binding domain thereof.

Most preferably the member of the TNFSF or receptor binding domain thereof is selected from CD40L or receptor binding domain thereof.

### TNFSF Members

The TNFSF comprises a number of structurally related members (also referred to as ligands) that organize lymphoid tissue development, co-stimulate lymphocyte activation and can either increase lymphocyte survival and function or induce cell death, all through bonding to their cognate receptor(s), which form the tumor necrosis factor receptor superfamily (TNFRSF).

TNFRSF may be divided into two distinct categories, depending on their ability to be activated by soluble ligand trimers of the TNFSF (Kucka K, 2021, Front Cell Dev Biol. 11; 8:615141).

Category I receptors of the TNFRSF are robustly activated by soluble ligand trimers, and include BaffR (ligand=Baff), DR3 (ligand=TL1A), GITR (ligand=GITRL), LTβR (ligand=LTαβ2 or LIGHT), TNFR1 (ligand=TNF or LTα).

Category II receptors of the TNFRSF fail to become properly activated by soluble ligand trimers despite high affinity binding, and include CD40L, 4-1BBL, Baff, APRIL, EDA, OX40L, CD70, TWEAK, FASL, TRAIL, and TNF.

The limited responsiveness of category II TNFRs to soluble TNFLs can be overcome by physical linkage of two or more soluble ligand trimers or, alternatively, by anchoring the soluble ligand molecules to the cell surface or extracellular matrix. In the context of the present invention, members of the TNFSF binding to category II TNFRs are preferred as the ability of the fusion protein according to the invention is then truly conditional, as activation of their cognate TNFR then depends on the presence in the tumor microenvironment of PD-L1 positive tumor cells.

Therefore, in a preferred embodiment, the member of the TNFSF included in the fusion protein according to the invention is selected from category II TNFSF members, preferably selected from CD40L, 4-1BBL, Baff, APRIL, EDA, OX40L, CD70, TWEAK, FASL, TRAIL, and TNF or receptor binding domain thereof.

TNFSF members may also be classified depending on their known functions. In the context of the present invention, TNFSF members involved in immune cell activation may preferably be included in the fusion protein according to the invention. TNFSF members involved in immune cell activation include CD40L, 4-1BBL, GITRL, OX40L, CD70, TL1A or receptor binding domain thereof (Croft M. et al., 2017, Nat Rev Rheumatol.; 13(4):217-233).

“CD40L”, “CD40 ligand”, “CD40LG”, “tumor necrosis factor superfamily member 5”, “TNFSF5”, and “CD154” are used herein interchangeably and refer to a member of the TNFSF that plays a central role in the initiation of adaptive immune response when it assembles into a homotrimer and interacts in trans with its receptor CD40. The CD40L-CD40 interaction leads to the activation of CD40 bearing cells, which then express adhesion (ICAM), co-stimulatory (CD80/CD86), and MHC I and II molecules in addition of cytokines/chemokines (TNFα, IL6 . . . ). In the tumor microenvironment, adhesion molecules and cytokine/chemokines act together to induce the infiltration and activation of immune cells, that ultimately destroy tumor cells and skew the tumor from an immunosuppressive to an immunocompetent microenvironment (Richards et al., 2020, Hum Vaccin Immunother. 16(2):377-387). CD40L is thus a TNFSF member involved in immune cell activation.

As TNF, CD40L assembles into a homotrimer and interacts in trans with its receptor CD40 through its extracellular part. CD40 is a category 11 receptor of the TNFRSF.

Being a member of the TNFSF that is both involved in immune cell activation and binds to a category 11 receptor of the TNFRSF, CD40L is a particularly preferred TNFSF member for the fusion protein according to the invention.

The specific CD40L protein that may be comprised in the fusion protein according to the invention is preferably selected from the same species as the species for which the fusion protein is intended for therapeutic use. In particular, for an intended use in humans, human CD40L will preferably be used. Human CD40L corresponds to Entrez Gene ID 959, and the complete amino acid sequence of human CD40L may be found under GenBank Accession No. NP_000065.1 (version of Jan. 17, 2022).

“4-1BBL”, “4-1BB ligand”, “CD137L”, “tumor necrosis factor superfamily member 9”, and “TNFSF9” are used herein interchangeably and refer to a transmembrane cytokine that acts as a ligand for TNFRSF9/4-1BB, which is a costimulatory receptor molecule in T lymphocytes. This cytokine and its receptor are involved in the antigen presentation process and in the generation of cytotoxic T cells. 4-1BBL is thus a TNFSF member involved in immune cell activation.

Its receptor 4-1BB is a category 11 receptor of the TNFRSF.

Being a member of the TNFSF that is both involved in immune cell activation and binds to a category II receptor of the TNFRSF, 4-1BBL is a particularly preferred TNFSF member for the fusion protein according to the invention.

The specific 4-1BBL protein that may be comprised in the fusion protein according to the invention is preferably selected from the same species as the species for which the fusion protein is intended for therapeutic use. In particular, for an intended use in humans, human 4-1BBL will preferably be used. Human 4-1BBL corresponds to Entrez Gene ID 8744, and the complete amino acid sequence of human 4-1BBL may be found under GenBank Accession No. NP_003802.1 (version of Feb. 27, 2022).

“OX40L”, “OX40 ligand”, “CD252”, “CD134L”, “tumor necrosis factor superfamily member 4”, and “TNFSF4” are used herein interchangeably and refer to an inducible molecule expressed on several cell types, although arguably most importantly, on antigen-presenting cells (APCs). OX40L can trigger signaling through its receptor OX40, resulting in a range of activities including expansion and accumulation of effector T cells and their cytokine production. OX40L is thus a TNFSF member involved in immune cell activation.

Its receptor OX40 is a category II receptor of the TNFRSF.

Being a member of the TNFSF that is both involved in immune cell activation and binds to a category II receptor of the TNFRSF, OX40L is a particularly preferred TNFSF member for the fusion protein according to the invention.

The specific OX40L protein that may be comprised in the fusion protein according to the invention is preferably selected from the same species as the species for which the fusion protein is intended for therapeutic use. In particular, for an intended use in humans, human OX40L will preferably be used. Human OX40L corresponds to Entrez Gene ID 7292, and the complete amino acid sequence of the longest isoform of human OX40L may be found under GenBank Accession No. NP_003317.1 (version of Mar. 16, 2022).

“CD70”, “CD27L”, “CD27LG”, “tumor necrosis factor superfamily member 7”, and “TNFSF7” are used herein interchangeably and refer to a molecule that can, through interaction with its receptor CD27, provide signals to T cells to control their accumulation and reactivity, similarly to that seen with OX40, GITR and DR3. CD70 is thus a TNFSF member involved in immune cell activation.

Its receptor CD27 is a category II receptor of the TNFRSF.

Being a member of the TNFSF that is both involved in immune cell activation and binds to a category II receptor of the TNFRSF, CD70 is a particularly preferred TNFSF member for the fusion protein according to the invention.

The specific CD70 protein that may be comprised in the fusion protein according to the invention is preferably selected from the same species as the species for which the fusion protein is intended for therapeutic use. In particular, for an intended use in humans, human CD70 will preferably be used. Human CD70 corresponds to Entrez Gene ID 970, and the complete amino acid sequences of the longest isoform of human CD70 may be found under GenBank Accession No. NP_001317261.1 (version of Jan. 9, 2022).

“Baff”, “B cell activating factor”, “CD257”, “tumor necrosis factor superfamily member 13b”, “TNFSF13B”, “tumor necrosis factor superfamily member 20”, and “TNFSF20” are used herein interchangeably and refer to a molecule that primarily, although not exclusively, controls B cell activity.

It binds to two receptors of the TNFRSF: BaffR and TACI. BaffR is a category I receptor of the TNFRSF, but TACI is a category II receptor of the TNFRSF.

Being a member of the TNFSF that binds to a category II receptor of the TNFRSF (TACI), Baff is a preferred TNFSF member for the fusion protein according to the invention.

The specific Baff protein that may be comprised in the fusion protein according to the invention is preferably selected from the same species as the species for which the fusion protein is intended for therapeutic use. In particular, for an intended use in humans, human Baff will preferably be used. Human Baff corresponds to Entrez Gene ID 10673, and the complete amino acid sequences of the longest isoform of human Baff may be found under GenBank Accession No. NP_006564.1 (version of Feb. 20, 2022).

“APRIL”, “CD256”, “tumor necrosis factor superfamily member 13”, and “TNFSF13” are used herein interchangeably and refer to a ligand found to be important for B cell development.

It binds to two receptors of the TNFRSF: BCMA and TACI, which are both category II receptors of the TNFRSF.

Being a member of the TNFSF that binds to category II receptors of the TNFRSF, APRIL is a preferred TNFSF member for the fusion protein according to the invention.

The specific APRIL protein that may be comprised in the fusion protein according to the invention is preferably selected from the same species as the species for which the fusion protein is intended for therapeutic use. In particular, for an intended use in humans, human APRIL will preferably be used. Human APRIL corresponds to Entrez Gene ID 8741, and the complete amino acid sequences of the longest isoform of human APRIL may be found under GenBank Accession No. NP_003799.1 (version of Jan. 23, 2022).

“EDA-A1”, “EDA-A2”, “EDA”, “ectodysplasin A”, and “tumor necrosis factor ligand 7C” are used herein interchangeably and refer to a protein involved in cell-cell signaling during the development of ectodermal organs.

Its receptor EDAR is a category II receptor of the TNFRSF.

Being a member of the TNFSF that binds to a category II receptor of the TNFRSF, EDA is a preferred TNFSF member for the fusion protein according to the invention.

The specific EDA protein that may be comprised in the fusion protein according to the invention is preferably selected from the same species as the species for which the fusion protein is intended for therapeutic use. In particular, for an intended use in humans, human EDA will preferably be used. Human EDA corresponds to Entrez Gene ID 1896, and the complete amino acid sequence of the longest isoform of human EDA may be found under GenBank Accession No. NP_001390.1 (version of Feb. 13, 2022).

“GITRL”, “Glucocorticoid-induced TNF receptor-related ligand”, “tumor necrosis factor superfamily member 18”, and “TNFSF18” are used herein interchangeably and refer to an inducible molecule expressed in professional APCs, and other cell types such as endothelial cells. Its receptor, glucocorticoid-induced TNF receptor-related protein (GITR, also known as TNFRSF18), can stimulate T cell, dendritic cell and B cell activation. GITRL is thus a TNFSF member involved in immune cell activation.

Its receptor GITR is a category I receptor of the TNFRSF.

Being a member of the TNFSF that is involved in immune cell activation, GITRL is a preferred TNFSF member for the fusion protein according to the invention.

The specific GITRL protein that may be comprised in the fusion protein according to the invention is preferably selected from the same species as the species for which the fusion protein is intended for therapeutic use. In particular, for an intended use in humans, human GITRL will preferably be used. Human GITRL corresponds to Entrez Gene ID 8995, and the complete amino acid sequences of the longest isoform of human GITRL may be found under GenBank Accession No. NP_005083.3 (version of Feb. 20, 2022).

“TL1A”, “VEGI”, “tumor necrosis factor superfamily member 15”, and “TNFSF15” are used herein interchangeably and refer to a protein that can be induced in APCs such as dendritic cells and macrophages, as well as in endothelial cells. Its binding to its receptor “Death receptor 3” (“DR3”, also known as “TNFRSF25”), a stimulatory receptor expressed by T cells, can regulate effector T cell accumulation and/or reactivity. TL1A is thus a TNFSF member involved in immune cell activation. This cytokine is also found to inhibit endothelial cell proliferation, and thus may function as an angiogenesis inhibitor.

Its receptor DR3 is a category I receptor of the TNFRSF.

Being a member of the TNFSF that is involved in immune cell activation, TL1A is a preferred TNFSF member for the fusion protein according to the invention.

The specific TL1A protein that may be comprised in the fusion protein according to the invention is preferably selected from the same species as the species for which the fusion protein is intended for therapeutic use. In particular, for an intended use in humans, human TL1A will preferably be used. Human TL1A corresponds to Entrez Gene ID 9966, and the complete amino acid sequences of the longest isoform of human TL1A may be found under GenBank Accession No. NP_005109.2 (version of Feb. 27, 2022).

“LIGHT”, “CD258”, “tumor necrosis factor superfamily member 14”, and “TNFSF14” are used herein interchangeably and refer to a protein that binds to two receptors of the TNFRSF: Herpes Virus Entry Mediator (HVEM, also known as tumor necrosis factor receptor superfamily member 14 (TNFRSF14)) and Lymphotoxin-β Receptor (LTβR). LIGHT-HVEM interaction is responsible for a majority of the immune-stimulating properties of LIGHT. Expressed on lymphocytes, NK cells, smooth muscle, and epithelium, HVEM serves as an important T cell costimulatory agent leading to activation, proliferation, and survival. In the context of anti-tumor immune support, LIGHT-LTβR signaling has a wide range of roles that span from influencing cancer cells' susceptibility to immune responses, functioning to repair chaotic tumor vasculature, and to supporting effector cells cell trafficking to and infiltration into tumors (Skeate Joseph G. et al. TNFSF14: LIGHTing the Way for Effective Cancer Immunotherapy. TNFSF14: LIGHTing the Way for Effective Cancer Immunotherapy. May 2020. Vol. 11. Article 922).

LTβR is a category I receptor of the TNFRSF. HVEM is a category I receptor of the TNFRSF.

The specific LIGHT protein that may be comprised in the fusion protein according to the invention is preferably selected from the same species as the species for which the fusion protein is intended for therapeutic use. In particular, for an intended use in humans, human LIGHT will preferably be used. Human LIGHT corresponds to Entrez Gene ID 8740, and the complete amino acid sequences of the longest isoform of human LIGHT may be found under GenBank Accession No. NP_003798.2 (version of Feb. 20, 2022).

“RANKL”, “RANK ligand”, “receptor activator of nuclear factor-KB ligand”, “CD254”, “tumor necrosis factor superfamily member 11”, and “TNFSF11” are used herein interchangeably and refer to a ligand for osteoprotegerin that functions as a key factor for osteoclast differentiation and activation. This protein was also shown to be a dentritic cell survival factor and to be involved in the regulation of T cell-dependent immune response.

Its receptor RANK (or “receptor activator of nuclear factor-KB”) is a category I receptor of the TNFRSF.

The specific RANKL protein that may be comprised in the fusion protein according to the invention is preferably selected from the same species as the species for which the fusion protein is intended for therapeutic use. In particular, for an intended use in humans, human RANKL will preferably be used. Human RANKL corresponds to Entrez Gene ID 8600, and the complete amino acid sequences of the longest isoform of human RANKL may be found under GenBank Accession No. NP_003692.1 (version of Feb. 27, 2022).

“TWEAK”, “tumor necrosis factor superfamily member 12”, and “TNFSF12” are used herein interchangeably and refer to a cytokine that has overlapping signaling functions with TNF, but displays a much wider tissue distribution. It exists in both membrane-bound and secreted forms, can induce apoptosis via multiple pathways of cell death in a cell type-specific manner. It is also found to promote proliferation and migration of endothelial cells, and thus acts as a regulator of angiogenesis.

Its receptor FN14 (also referred to as TWEAKR) is a category II receptor of the TNFRSF.

Being a member of the TNFSF that binds to a category II receptor of the TNFRSF, TWEAK is a preferred TNFSF member for the fusion protein according to the invention.

The specific TWEAK protein that may be comprised in the fusion protein according to the invention is preferably selected from the same species as the species for which the fusion protein is intended for therapeutic use. In particular, for an intended use in humans, human TWEAK will preferably be used. Human TWEAK corresponds to Entrez Gene ID 8742, and the complete amino acid sequence of the longest isoform of human TWEAK may be found under GenBank Accession No. NP_003800.1 (version of Feb. 20, 2022).

“FASL”, “FAS ligand”, “FASLG”, “CD178”, “CD95 ligand”, “CD95L”, “tumor necrosis factor superfamily member 6”, and “TNFSF6” are used herein interchangeably and refer to a transmembrane protein which primary function is the induction of apoptosis triggered by binding to its receptor FAS. The FAS/FASLG signaling pathway is essential for immune system regulation, including activation-induced cell death (AICD) of T cells and cytotoxic T lymphocyte induced cell death.

FAS is a category II receptor of the TNFRSF.

Being a member of the TNFSF that binds to a category II receptor of the TNFRSF, FASL is a preferred TNFSF member for the fusion protein according to the invention.

The specific FASL protein that may be comprised in the fusion protein according to the invention is preferably selected from the same species as the species for which the fusion protein is intended for therapeutic use. In particular, for an intended use in humans, human FASL will preferably be used. Human FASL corresponds to Entrez Gene ID 356, and the complete amino acid sequence of the longest isoform of human FASL may be found under GenBank Accession No. NP_000630.1 (version of Feb. 27, 2022).

“TRAIL”, “CD253”, “tumor necrosis factor superfamily member 10”, and “TNFSF10” are used herein interchangeably and refer to a protein that preferentially induces apoptosis in transformed and tumor cells, but does not appear to kill normal cells although it is expressed at a significant level in most normal tissues. TRAIL binds to several members of TNF receptor superfamily including TNFRSF10A/TRAILR1, TNFRSF10B/TRAILR2, TNFRSF10C/TRAILR3, TNFRSF10D/TRAILR4, and possibly also to TNFRSF11B/OPG. The activity of TRAIL may be modulated by binding to the decoy receptors TNFRSF10C/TRAILR3, TNFRSF10D/TRAILR4, and TNFRSF11B/OPG that cannot induce apoptosis. The binding of TRAIL to its receptors has been shown to trigger the activation of MAPK8/JNK, caspase 8, and caspase 3.

Both of TRAILR1 and TRAILR2 are category II receptors of the TNFRSF.

Being a member of the TNFSF that binds to category II receptors of the TNFRSF, TRAIL is a preferred TNFSF member for the fusion protein according to the invention.

The specific TRAIL protein that may be comprised in the fusion protein according to the invention is preferably selected from the same species as the species for which the fusion protein is intended for therapeutic use. In particular, for an intended use in humans, human TRAIL will preferably be used. Human TRAIL corresponds to Entrez Gene ID 8743, and the complete amino acid sequence of the longest isoform of human TRAIL may be found under GenBank Accession No NP_003801.1 (version of Mar. 17, 2022).

“TNF”, “tumor necrosis factor”, “TNFA”, “TNFα”, “tumor necrosis factor superfamily member 2”, and “TNFSF2” are used herein interchangeably and refer to a multifunctional proinflammatory cytokine involved in the regulation of a wide spectrum of biological processes including cell proliferation, differentiation, apoptosis, lipid metabolism, and coagulation.

TNF binds to two receptors of the TNFRSF: TNFR1 (also known as “TNFRSF1A”) and TNFR2 (also known as “TNFRSF1B” or “TNFBR”). TNFR1 is a category I receptor of the TNFRSF, while TNFR2 is a category II receptor of the TNFRSF.

Being a member of the TNFSF that binds to a category II receptor of the TNFRSF, TNF is a preferred TNFSF member for the fusion protein according to the invention.

The specific TNF protein that may be comprised in the fusion protein according to the invention is preferably selected from the same species as the species for which the fusion protein is intended for therapeutic use. In particular, for an intended use in humans, human TNF will preferably be used. Human TNF corresponds to Entrez Gene ID 7124, and the complete amino acid sequence of human TNF may be found under GenBank Accession No NP_000585.2 (version of Mar. 17, 2022).

“CD30L”, “CD153”, “TNFSF8”, and “tumor-necrosis factor superfamily member 8” are used herein interchangeably and refer to a protein that display restricted expression in subpopulations of activated T- and B-cells in non-pathologic conditions and regulates proliferation/apoptosis and antibody responses.

Its receptor TNFRSF8 (also referred to as Tumor necrosis factor receptor superfamily member 8 or CD30) is a category I receptor of the TNFRSF.

The specific CD30L protein that may be comprised in the fusion protein according to the invention is preferably selected from the same species for which the fusion protein is intended for therapeutic use. In particular, for an intended use in humans, human CD30L will preferably be used. Human CD30L corresponds to Entrez Gene ID 943, and the complete amino acid sequence of human CD30L may be found under GenBank Accession No AH005843.2 (version of Jun. 10, 2016).

“LTα”, “lymphotoxin alpha”, “TNFB”, “TNFP”, “tumor necrosis factor superfamily member 1”, and “TNFSF1” are used herein interchangeably and refer to a cytokine produced by lymphocytes that is highly inducible, secreted, and forms heterotrimers with lymphotoxin-beta which anchor lymphotoxin-alpha to the cell surface. This protein also mediates a large variety of inflammatory, immunostimulatory, and antiviral responses, is involved in the formation of secondary lymphoid organs during development and plays a role in apoptosis.

LTα binds to TNFR1 (also known as “TNFRSF1A”), which is a category I receptor of the TNFRSF.

The specific LTα protein that may be comprised in the fusion protein according to the invention is preferably selected from the same species as the species for which the fusion protein is intended for therapeutic use. In particular, for an intended use in humans, human LTα will preferably be used. Human LTα corresponds to Entrez Gene ID 4049, and the complete amino acid sequence of human LTα may be found under GenBank Accession No NP_001153212.1 (version of Mar. 10, 2022).

“LTβ”, “lymphotoxin beta”, “TNFC”, “tumor necrosis factor superfamily member 3”, and “TNFSF3” are used herein interchangeably and refer to a protein that anchors lymphotoxin-alpha to the cell surface through heterotrimer formation. The predominant form on the lymphocyte surface is the lymphotoxin-alpha 1/beta 2 complex (also referred to as “LTαβ2”, e.g. 1 molecule alpha/2 molecules beta) and this complex is the primary ligand for the lymphotoxin-beta receptor (also referred to as LTβR”), a category I receptor of the TNFRSF. The minor complex is lymphotoxin-alpha 2/beta 1. LTB is an inducer of the inflammatory response system and involved in normal development of lymphoid tissue.

The specific LTβ protein that may be comprised in the fusion protein according to the invention is preferably selected from the same species as the species for which the fusion protein is intended for therapeutic use. In particular, for an intended use in humans, human LTβ will preferably be used. Human LTβ corresponds to Entrez Gene ID 4050, and the complete amino acid sequence of human LTβ may be found under GenBank Accession No NP_002332.1 (version of Feb. 20, 2021).

### Functional Fragments or Variants of TNFSF Members

While the SPD-TNFSF fusion protein according to the invention may comprise an entire wild-type member of the TNFSF, it may also alternatively comprise a functional fragment thereof or a functional derivative thereof.

By a “functional fragment” of a member of the TNFSF, it is meant a fragment (i.e. a part of the amino acid sequence of the entire member of the TNFSF) with one or more insertions, deletions, truncations and/or substitutions, and that retains the function of the entire member of the TNFSF, i.e. its ability to trimerize and bind to and activate its TNFRSF receptor(s).

In particular, TNFSF members exist as transmembrane proteins, but their transmembrane and intracellular regions are not needed for trimerization and binding to and activation of their TNFRSF receptor(s).

Therefore, in a preferred embodiment, the TNFSF member fragment comprised in the SPD-TNFSF fusion protein according to the invention is an extracellular fragment of the TNFSF member, i.e. a fragment devoid of the transmembrane and intracellular part of the TNFSF member. Whole or part of the extracellular domain may be comprised in the extracellular fragment, provided that the fragment retains its ability to trimerize and bind to and activate its TNFRSF receptor(s).

In a preferred embodiment, the TNFSF ligand or receptor binding thereof of the fusion protein is selected from human CD40L (SEQ ID NO:24), particularly amino acids 119-261 of human CD40L (SEQ ID NO:25).

In a further preferred embodiment, the TNFSF ligand or receptor binding thereof of the fusion protein is selected from murine CD40L (SEQ ID NO:26), particularly amino acids 115-260 of murine CD40L (SEQ ID NO:27).

In another embodiment, the TNFSF ligand or receptor binding thereof of the fusion protein is selected from human 4-1-BBL (SEQ ID NO:28), particularly amino acids 80-254 of human 4-1-BBL (SEQ ID NO:29).

In still another embodiment, the TNFSF ligand or receptor binding thereof of the fusion protein is selected from murine 4-1-BBL (SEQ ID NO:30), particularly amino acids 139-309 of murine 4-1-BBL (SEQ ID NO:31).

In one embodiment, the SPD-TNFSF fusion protein comprises a collagen domain. Typically, the collagen domain comprises or consists of between 1 and 40 (GXX) repeats, preferably between 3 and 30 (GXX) repeats, preferably between 6 and 20 (GXX) repeats, more preferably 12 (GXX) repeats, wherein X is an amino acid, and G is a glycine amino acid, and wherein each X may be identical or different in each repeat. The collagen domain may be located between the N-terminus domain and the coiled-coil neck domain of SPD. Examples of suitable collagen domain are presented in Table 1 below:

TABLE 1
Collagen domain and corresponding number of (GXX) repeats. By “6 −
N-Gly” it is to be understood, the considered collagen domain
comprises 6 (GXX) repeats without a N-Glycosylation site, and
by “6 + N-Gly”, the considered collagen domain comprises
6 (GXX) repeats including a N-Glycosylation site. By “12”
or “12bis”, it is to be understood the considered collagen
domain comprises 12 (GXX) repeats, however “12” and “12bis”
present different sequences.

| | Number of | SEQ ID NO |
|---|---|---|
| Name | (GXX) repeats | reference |
| Collagen domain(6 − N-Gly) | 6 | 32 |
| Collagen domain(6 + N-Gly) | 6 | 33 |
| Collagen domain(12) | 12 | 34 |
| Collagen domain(12bis) | 12 | 35 |
| Collagen domain(19) | 19 | 36 |
| Collagen domain(30) | 30 | 37 |

In one embodiment, the SPD-TNFSF fusion protein as described herein may additionally comprise a linker located between the coiled-coil neck domain and the TNFSF ligand or the receptor binding domain thereof. Typically, linkers are composed of a short stretch of amino acid residues such as glycine (Gly or G), serine (Ser or S), Threonine (Thr or T), asparagine (Asn or N), alanine (Ala or A) and/or proline (Pro or P). The said linker is preferably a glycine/serine linker, i.e. constituted essentially of glycine and serine amino acids. The said linker has preferably a length of 4-20 amino acids, particularly 4, 8, 12, 16 or 20 amino acids (e.g. 1, 2 3 or 4 repetitions of GGGS, GSGSG, or SGSGS, or 1 or 2 repetitions of GSGSGSGSGS). More preferably, the length of the linker is 8 to 16 amino acids, and even more preferably 12 amino acids. For guidance, a suitable linker for use in the context of the present invention comprises the amino acid sequence shown in SEQ ID NO:38 (GGGSGGGSGGGS). It is within the reach of the skilled person to optimize the size and sequence of a peptide linker between the two fusion partners.

In one embodiment, the SPD-TNFSF fusion protein as described herein comprises a collagen domain and a linker respectively located at the N-terminus and C-Terminus of the coiled-coil neck domain. Suitable SPD-TNFSF fusion protein comprises the amino acid sequence shown in SEQ ID NO:11.

In the context of the invention, it may be advantageous to include additional regulatory elements to facilitate expression, trafficking and biological activity of SPD-TNFSF fusion protein.

In the context of the present invention, SPD-TNFSF fusion protein may additionally comprise a signal peptide at the N-terminus of the SPD-TNFSF fusion protein to affect the expression levels, secretion, solubility, or other property of the protein. Appropriate signal peptides are known in the art. They may be obtained from cellular or viral polypeptides such as those of immunoglobulins, tissue plasminogen activator, insulin, rabies glycoprotein, the HIV virus envelope glycoprotein or the measles virus F protein or may be synthetic (see e.g. WO2008/138649). For guidance, a suitable signal peptide for use in the context of the present invention comprises the amino acid sequence shown in SEQ ID NO:39 (MLLFLLSALVLLTQPLGYLE), SEQ ID NO:40 (MGLGLQWVFFVALLKGVHC) or in SEQ ID NO:41 (MGWSCIILFLVATATGVHS). One may also envisage addition of transmembrane domain to facilitate anchorage of the TNFSF fusion protein in a suitable membrane (e.g. the plasmic membrane) of the cells. The transmembrane domain is typically inserted at the N-terminus or C-terminus of the protein. A vast variety of transmembrane domains are known in the art (see e.g. WO99/03885).

In the context of the present invention, SPD-TNFSF fusion protein may also additionally comprise a tag peptide (typically a short peptide sequence able to be recognized by available antisera or compounds) for following expression, trafficking, or purification of the SPD-TNFSF protein or of infected host cells expressing such fusion protein. Tag peptides can be detected by immunodetection assays using anti-tag antibodies. A vast variety of tag peptides can be used in the context of the invention including, without limitation, PK tag, FLAG (DYKDDDK, SEQ ID NO:42, GDYKDDDK, SEQ ID NO:43, GSDYKDDDDK, SEQ ID NO:44 or HHHHHHDYKDDDDKLVPRGS, SEQ ID NO:45), MYC tag (QKLISEEDL, SEQ ID NO:46), HIS tag (usually a stretch of 4 to 10 histidine residues), HA tag (YPYDVPDYA; SEQ ID NO:47), HSV tag (QPELAPEDPED; SEQ ID NO:48), VSV Tag (YTDIEMNRLGK; SEQ ID NO:49) and e-tag (U.S. Pat. No. 6,686,152). For guidance, a suitable tag peptide for use in the context of the present invention comprises the amino acid sequence shown in SEQ ID NO:42, SEQ ID NO:43, SEQ ID NO:44 or SEQ ID NO:45. The tag peptide(s) may be independently positioned at the N-terminus of the protein or alternatively at its C-terminus or alternatively internally or at any of these positions when several tags are employed. When a signal peptide is already present in N-terminal of the fusion protein, a tag peptide may preferably be inserted at the C-terminus of the fusion protein according to the invention.

As another example, the glycosylation can be altered so as to increase biological activity of SPD-TNFSF fusion protein. Such modifications can be accomplished, for example, by mutating one or more residues within the site(s) of glycosylation.

The terms “oligomeric” or “multimeric” as used herein refers to the ability of at least two monomeric units to form a complex. The association can be specific (requiring a structural complementarity between amino acid residues of the two partners at a binding site and one or more type(s) of electrostatic forces, hydrogen bonding, hydrophobic forces, and/or van der Waals forces to maintain the binding or non-specific (interaction through one or more type(s) of the above cited forces but lacking the structural complementarity). Oligomers of SPD-TNFSF fusion protein are preferably formed by one or more intermolecular disulfide bonding involving one or more cysteine (Cys) residues on each polypeptide forming the oligomer such that disulfide bond(s) can form between the oligomerized proteins (i.e. intermolecular disulfide bonds). Preferably, the oligomer is formed between at least two SPD-TNFSF fusion proteins (dimer), three SPD-TNFSF fusion proteins (trimer), six SPD-TNFSF fusion proteins or two trimers of SPD-TNFSF fusion protein (hexamer), twelve SPD-TNFSF fusion proteins (dodecamer), eighteen SPD-TNFSF fusion proteins (octadecamer) or strictly more than eighteen SPD-TNFSF fusion proteins (highly order oligomer).

The fusion protein may be a monomeric protein or a multimeric protein. Preferably the fusion protein is present as a multimeric form consisting of three SPD-TNFSF fusion proteins self-assembled in trimeric SPD-TNFSF fusion protein which may be identical or different. Preferably, the multimeric form is a dodecameric form consisting of assembly of four identical trimeric forms.

### Oncolytic Virus

The oncolytic virus of the present invention can be obtained from any member of virus identified at present time provided that it is oncolytic by its propensity to selectivity replicate and kill dividing cells as compared to non-dividing cells. It may be a native virus that is naturally oncolytic or may be engineered by modifying one or more viral genes so as to increase tumor selectivity and/or preferential replication in dividing cells, such as those involved in DNA replication, nucleic acid metabolism, host tropism, surface attachment, virulence, lysis and spread (see for example Kirn et al., 2001, Nat. Med. 7: 781; Wong et al., 2010, Viruses 2: 78-106). One may also envisage placing one or more viral gene(s) under the control of event or tissue-specific regulatory elements (e.g. promoter).

Exemplary oncolytic viruses include without limitation reovirus, Seneca Valley virus (SVV), vesicular stomatitis virus (VSV), Newcastle disease virus (NDV), herpes simplex virus (HSV), morbillivirus, retrovirus, influenza virus, Sinbis virus, poxvirus, adenovirus, adenovirus-associated virus (AAV), measles virus, foamy virus, alpha virus, lentivirus, rhabdovirus, picornavirus, coxsackievirus, parvovirus or chimeras thereof.

In one embodiment, the oncolytic virus of the present invention is obtained from a reovirus. A representative example includes Reolysin (under development by Oncolytics Biotech; NCT01166542).

In one embodiment, the oncolytic virus of the present invention is obtained from a Seneca Valley virus. A representative example includes NTX-010 (Rudin et al., 2011, Clin. Cancer. Res. 17(4): 888-95).

In one embodiment, the oncolytic virus of the present invention is obtained from a vesicular stomatitis virus (VSV). Representative examples are described in the literature (e.g. Stojdl et al., 2000, Nat. Med. 6(7): 821-5; Stojdl et al., 2003, Cancer Cell 4(4): 263-75).

In one embodiment, the oncolytic virus of the present invention is obtained from a Newcastle disease virus. Representative examples include without limitation the 73-T PV701 and HDV-HUJ strains as well as those described in the literature (e.g. Phuangsab et al., 2001, Cancer Lett. 172(1): 27-36; Lorence et al., 2007, Curr. Cancer Drug Targets 7(2): 157-67; Freeman et al., 2006, Mol. Ther. 13(1): 221-8).

In one embodiment, the oncolytic virus of the present invention is obtained from a herpes virus. The Herpesviridae are a large family of DNA viruses that all share a common structure and are composed of relatively large double-stranded, linear DNA genomes encoding 100-200 genes encapsided within an icosahedral capsid which is enveloped in a lipid bilayer membrane. Although the oncolytic herpes virus can be derived from different types of HSV, particularly preferred are HSV1 and HSV2. The herpes virus may be genetically modified so as to restrict viral replication in tumors or reduce its cytotoxicity in non-dividing cells. For example, any viral gene involved in nucleic acid metabolism may be inactivated, such as genes encoding thymidine kinase (Martuza et al., 1991, Science 252: 854-6), ribonucleotide reductase (RR) (Boviatsis et al., Gene Ther. 1: 323-31; Mineta et al., 1994, Cancer Res. 54: 3363-66), or uracil-N-glycosylase (Pyles et al., 1994, J. Virol. 68: 4963-72). Another aspect involves viral mutants with defects in the function of genes encoding virulence factors such as the ICP34.5 gene (Chambers et al., 1995, Proc. Natl. Acad. Sci. USA 92: 1411-5). Representative examples of oncolytic herpes virus include NV1020 (e.g. Geevarghese et al., 2010, Hum. Gene Ther. 21(9): 1119-28) and T-VEC (Andtbacka et al., 2013, J. Clin. Oncol. 31, abstract number LBA9008).

In one embodiment, the oncolytic virus of the present invention is obtained from a morbillivirus which can be obtained from the paramyxoviridae family, with a specific preference for measles virus. Representative examples of oncolytic measles viruses include without limitation MV-Edm (McDonald et al., 2006; Breast Cancer Treat. 99(2): 177-84) and HMWMAA (Kaufmann et al., 2013, J. Invest. Dermatol. 133(4): 1034-42)

In one embodiment, the oncolytic virus of the present invention is obtained from an adenovirus. Methods are available in the art to engineer oncolytic adenoviruses. An advantageous strategy includes the replacement of viral promoters with tumor-selective promoters or modifications of the E1 adenoviral gene product(s) to inactivate its/their binding function with p53 or retinoblastoma (Rb) protein that are altered in tumor cells. In the natural context, the adenovirus E1B55 kDa gene cooperates with another adenoviral product to inactivate p53 (p53 is frequently dysregulated in cancer cells), thus preventing apoptosis. Representative examples of oncolytic adenovirus include ONYX-015 (e.g. Khuri et al., 2000, Nat. Med 6(8): 879-85) and H101 also named Oncorine (Xia et al., 2004, Ai Zheng 23(12): 1666-70).

In one embodiment, the oncolytic virus of the present invention is obtained from an adeno-associated virus serotype, including without limitation, AAV-1, AAV-2, AAV-3, AAV-4, AAV-5, AAV-6, AAV-7, AAV-8 etc. or any other virus or serotype which is substantially homologous in its capsid protein sequence to the AAV-2 or capsid protein sequence.

In one embodiment, the oncolytic virus of the present invention is a poxvirus. As used herein the term “poxvirus” refers to a virus belonging to the Poxviridae family, with a specific preference for a poxvirus belonging to the Chordopoxviridae subfamily and more preferably to the Orthopoxvirus genus or chimeras thereof. Sequences of the genome of various poxviruses, for example, the vaccinia virus, cowpox virus, Canarypox virus and Ectromelia virus genomes are available in the art and specialized databases such as Genbank (accession number NC_006998, NC_003663, NC_005309, NC_004105 respectively).

Advantageously, the oncolytic poxvirus is a cowpox virus and can derive from any cowpox strain, like for example, CPXV_GER1980_EP4 (Genbank HQ420895), CPXV_GER2002_MKY (Genbank HQ420898), CPXV_GER1991_3 (Genbank DQ 437593), CPXV_FRA2001_A CY (Genbank HQ420894), CPXV_GR1990_2 (Genbank HQ420896), CPXV_UK2000_K2984 (Genbank HQ420900), CPXV_BR (Genbank AF482758.2 or NC 003663) and CPXV_NOR1994-MAN (Genbank HQ420899), CPXV_GER1998_2 (Genbank HQ420897), CPXV_GRI (Genbank X94355), CPXV_FIN2000_MAN (Genbank HQ420893) and CPXV_AUS1999_867 (Genbank HQ407377).

Desirably, the oncolytic poxvirus is an oncolytic vaccinia virus. Vaccinia viruses are members of the poxvirus family characterized by a 200 kb double-stranded DNA genome that encodes numerous viral enzymes and factors that enable the virus to replicate independently from the host cell machinery. The majority of vaccinia virus particles is intracellular (IMV for intracellular mature virion) with a single lipid envelop and remains in the cytosol of infected cells until lysis. The other infectious form is a double enveloped particle (EEV for extracellular enveloped virion) that buds out from the infected cell without lysing it.

Although it can derive from any vaccinia virus strain, Elstree, Wyeth, Copenhagen, Lister, Tian Tian and Western Reserve strains are particularly preferred. The gene nomenclature used herein is that of Copenhagen vaccinia strain. It is also used herein for the homologous genes of other poxviridae unless otherwise indicated. However, gene nomenclature may be different according to the pox strain but correspondence between Copenhagen and other vaccinia strains are generally available in the literature.

In one embodiment, the poxvirus belonging to the Chordopoxviridae subfamily and more preferably to the Leporipoxvirus genus with a preference for myxoma virus, rabbit fibroma virus or squirrel fibroma virus and more preferably myxoma virus (which genomic sequences are disclosed in Genbank under accession number NP_051868.1).

Preferably, the oncolytic virus of the present invention is modified by altering for one or more viral gene(s). Said modification(s) preferably lead(s) to the synthesis of a defective protein unable to ensure the activity of the protein produced under normal conditions by the unmodified gene (or lack of synthesis). Modifications encompass deletion, mutation and/or substitution of one or more nucleotide(s) (contiguous or not) within the viral gene or its regulatory elements. Modification(s) can be made in a number of ways known to those skilled in the art using conventional recombinant techniques. Exemplary modifications are disclosed in the literature with a specific preference for those altering viral genes involved in DNA metabolism, host virulence, IFN pathway (see e.g. Guse et al., 2011, Expert Opinion Biol. Ther. 11(5): 595-608) and the like.

More preferably, the oncolytic poxvirus of the present invention is modified by altering the thymidine kinase-encoding gene (locus J2R). The TK enzyme is involved in the synthesis of deoxyribonucleotides. TK is needed for viral replication in normal cells as these cells have generally low concentration of nucleotides whereas it is dispensable in dividing cells which contain high nucleotide concentration.

Alternatively, or in combination, the oncolytic poxvirus of the present invention is modified by altering at least one gene or both genes encoding Ribonucleotide reductase (RR). In the natural context, this enzyme catalyses the reduction of ribonucleotides to deoxyribonucleotides that represents a crucial step in DNA biosynthesis. The viral enzyme is similar in subunit structure to the mammalian enzyme, being composed of two heterologous subunits, designed R1 and R2 encoded respectively by the I4L and F4L locus. Sequences for the I4L and F4L genes and their locations in the genome of various poxvirus are available in public databases, for example via accession number DQ437594, DQ437593, DQ377804, AH015635, AY313847, AY313848, NC_003391, NC_003389, NC_003310, M-35027, AY243312, DQ011157, DQ011156, DQ011155, DQ011154, DQ011153, Y16780, X71982, AF438165, U60315, AF410153, AF380138, U86916, L22579, NC_006998, DQ121394 and NC_008291. In the context of the invention, either the I4L gene (encoding the R1 large subunit) or the F4L gene (encoding the R2 small subunit) or both may be inactivated.

Alternatively to or in combination with, the oncolytic poxvirus may be further modified, in the M2L locus (preference for modification leading to a suppressed expression of the viral m2 protein), resulting in a modified poxvirus defective m2 functions (m2-defective poxvirus).

In one embodiment, the oncolytic poxvirus is further modified in the M2L locus and in the J2R locus (preference for modification resulting in a suppressed expression of the viral tk protein), resulting in an oncolytic poxvirus defective for both m2 and tk functions (m2−tk− poxvirus). Partial or complete deletion of said M2L locus and/or J2R locus as well as insertion of foreign nucleic acid in the M2L locus and/or J2R locus are contemplated in the context of the present invention to inactivate m2 and tk functions.

Alternatively to or in combination with, the oncolytic poxvirus may be further modified in the M2L locus and in the I4L and/or F4L locus/loci (preference for modification leading to a suppressed expression of the viral ribonucleotide reductase (rr) protein), resulting in a oncolytic poxvirus defective for both m2 and rr functions (m2 and rr-defective poxvirus). In the context of the invention, the poxvirus can be modified either in the I4L gene (encoding the r1 large subunit) or in the F4L gene (encoding the r2 small subunit) or both to provide a rr-defective poxvirus. e.g. by partial or complete deletion of said I4L and/or F4L locus/loci.

Also provided is a oncolytic poxvirus modified in the M2L locus, in the J2R locus, and in the I4L and/or F4L loci (triple defective virus with modifications in the M2L, J2R and I4L loci; M2L, J2R and F4L loci or M2L, J2R, I4L and F4L loci), resulting in a oncolytic poxvirus defective for m2, tk and rr activities (m2−, tk−rr− poxvirus).

Alternatively, or in combination, other strategies may also be pursued to further increase the virus tumor-specificity. A representative example of suitable modification includes disruption of the VGF-encoding gene from the viral genome. VGF (for VV growth factor) is a secreted protein which is expressed early after cell infection and its function seems important for virus spread in normal cells. Another example is the disruption of the A56R gene coding for hemagglutinin, eventually in combination with tk deletion (Zhang et al., 2007, Cancer Res. 67: 10038-46). Disruption of interferon modulating gene(s) may also be advantageous (e.g. the B8R or B18R gene) or the caspase-1 inhibitor B13R gene. Another suitable modification comprises the disruption of the F2L gene which encodes the viral dUTPase involved in both maintaining the fidelity of DNA replication and providing the precursor to produce TMP by thymidylate synthase (Broyles et al., 1993, Virol. 195: 863-5). Sequence of the vaccinia virus F2L gene is available in GenBank via accession number M25392.

In a preferred embodiment, the oncolytic virus of this invention is a vaccinia virus defective for TK activity resulting from inactivating mutations in the J2R gene. In another preferred embodiment, the oncolytic virus of this invention is a vaccinia virus defective for both TK and RR activities resulting from inactivating mutations in both the J2R gene and the I4L and/or F4L gene(s) carried by the viral genome (e.g. as described in WO2009/065546 and Foloppe et al., 2008, Gene Ther., 15: 1361-71). In a further preferred embodiment, the oncolytic virus of the invention is a vaccinia virus defective for TK, RR and m2 activities resulting for inactivating mutations in both the J2R gene, the I4L and/or F4L gene(s) and M2L gene. In another preferred embodiment, the oncolytic virus of this invention is a vaccinia virus defective for dUTPase resulting from inactivating mutations in the F2L gene (e.g. as described in WO2009/065547), eventually in combination with disruption of at least one of TK and RR activities or both (resulting in a virus with inactivating mutations in the F2L; F2L and J2R gene; F2L and I4L; or F2L, J2R and I4L).

### Non-Oncolytic Virus

In one embodiment, the non-oncolytic virus of the invention is a poxvirus. Exemplary non-oncolytic virus poxvirus includes without limitation Pseudocowpox virus (PCPV), Modified vaccinia virus Ankara (MVA), highly attenuated vaccinia virus strain (NYVAC), Swinepox virus (SWPV), Fowlpox virus (FPV) or chimeras thereof.

In one embodiment, the non-oncolytic poxvirus of the present invention is obtained from a Pseudocowpox virus (PCPV). Representative examples of suitable PCPV strains for use herein include, without limitation, YG2828 (GenBank accession number LC2301 19), F07.801 R (GenBank accession number JF773693), F10.3081 C (GenBank accession number JF773695), F07.798R (GenBank accession number JF773692), F99.177C (GenBank accession number AY453678), IT1303/05 (GenBank accession number JF800906), F00.120R (GenBank accession number GQ329669; Tikkanen et al., 2004, J. Gen. Virol. 85: 1413-8) and TJS (also called VR634; GenBank accession number GQ329670; Friedman-Kien et al., 1963, Science 140: 1335-6; available at ATCC under accession number VR634). Such strains may have morphological, structural and/or genetic differences each other, e.g., in terms of ITR length, number of predicted genes and/or G C rich content (see e.g. Hautaniemi et al., 2010, J. Gen. Virol. 91:1560-76). In a preferred embodiment, the PCPV virus of the present invention is obtained from the wild-type TJS strain as identified by ATCC reference number ATCC VR-634™ or from a virus strain of the same or similar name and functional fragments and variants thereof.

In one embodiment, the non-oncolytic poxvirus of the present invention is obtained from a highly attenuated vaccinia virus strain (NYVAC) (Tartaglia et al., 1992, Virol. 188(1):217-32, U.S. Pat. No. 5,494,807). For illustrative purpose, NYVAC is a highly attenuated vaccinia virus strain, derived from a plaque-cloned isolate of the Copenhagen vaccine strain by the precise deletion of 18 open reading frames (ORFs) from the viral genome.

In one embodiment, the non-oncolytic poxvirus of the present invention is obtained from modified Vaccinia virus Ankara (MVA), due to its highly attenuated phenotype (Mayr et al., 1975, Infection 3: 6-14; Sutter and Moss, 1992, Proc. Natl. Acad. Sci. USA 89: 10847-51). The nucleotide sequence of the MVA genome and amino sequence of the encoded viral proteins are available in the art, e.g. from Antoine et al. (1998, Virol, 244: 365-96) and GenBank (accession number U94848).

In one embodiment, the non-oncolytic poxvirus of the present invention is obtained from Swinepox virus (SWPV). The nucleotide sequence of the SWPV genome and amino sequence are available in the art, e.g. Alfonso et al. (2002, Virol, 76(2):783-90) and GenBank accession (NC_003389.1 and MW036632).

In one embodiment, the non-oncolytic poxvirus of the present invention is obtained from Fowlpox virus (FPV). The nucleotide sequence of Fowlpox virus genome is available in the art, e.g. Alfonso et al. (2000, Virol., 74(8): 3815-383) and GenBank accession (AF198100).

Expression of the One or More Nucleic Acid Molecule(s) Encoding the SPD-TNFSF Fusion Protein Inserted into the Viral Genome.

The SPD-TNFSF-encoding nucleic acid molecule(s) may be easily obtained by standard molecular biology techniques (e.g. PCR amplification, cDNA cloning, chemical synthesis) using sequence data accessible in the art and the information provided herein. Analogs and fragments may be generated using standard techniques of molecular biology.

In one embodiment, the nucleic acid molecule(s) encoding the SPD-TNFSF can independently be inserted at any location of the viral genome, with a specific preference for a non-essential locus. Insertion into the virus can be performed by routine molecular biology, e.g. as described in Sambrook et al. (2001, Molecular Cloning-A Laboratory Manual, Cold Spring Harbor Laboratory). Insertion into an adenoviral vector or a poxviral vector can be performed through homologous recombination as described respectively in Chartier et al. (1996, J. Virol. 70: 4805-10) and Paul et al. (2002, Cancer gene Ther. 9: 470-7). For example, TK, RR and F2L genes as well as intergenic regions are particularly appropriate for insertion in oncolytic vaccinia virus and E3 and E4 regions are appropriate for insertion in oncolytic adenovirus.

In addition, the encoding nucleotide sequences can be optimized for providing high level expression in a particular host cell or subject. It has been indeed observed that, the codon usage patterns of organisms are highly non-random and the use of codons may be markedly different between different hosts. For example, the therapeutic gene may be from bacterial or lower eukaryote origin (e.g. the suicide gene), and thus have an inappropriate codon usage pattern for efficient expression in higher eukaryotic cells (e.g. human). Typically, codon optimization is performed by replacing one or more “native” (e.g. bacterial or yeast) codon corresponding to a codon infrequently used in the host organism of interest by one or more codon encoding the same amino acid which is more frequently used. It is not necessary to replace all native codons corresponding to infrequently used codons since increased expression can be achieved even with partial replacement.

Further to optimization of the codon usage, expression in the host cell or subject can further be improved through additional modifications of the nucleotide sequence(s). For example, various modifications may be envisaged so as to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; R A secondary structures; and/or internal cryptic regulatory elements such as internal TATA-boxes, chi-sites, ribosome entry sites, and/or splicing donor/acceptor sites.

In accordance with the present invention, each of the one or more nucleic acid molecule(s) encoding said SPD-TNFSF fusion protein inserted in the genome of the virus of the invention is operably linked to suitable regulatory elements for its expression in a host cell or subject. As used herein, the term “regulatory elements” or “regulatory sequence” refers to any element that allows, contributes or modulates the expression of the encoding nucleic acid molecule(s) in a given host cell or subject, including replication, duplication, transcription, splicing, translation, stability and/or transport of the nucleic acid(s) or its derivative (i.e. m RNA). As used herein, “operably linked” means that the elements being linked are arranged so that they function in concert for their intended purposes. For example, a promoter is operably linked to a nucleic acid molecule if the promoter effects transcription from the transcription initiation to the terminator of said nucleic acid molecule in a permissive host cell.

It will be appreciated by those skilled in the art that the choice of the regulatory sequences can depend on such factors as the nucleic acid molecule itself, the virus into which it is inserted, the host cell or subject, the level of expression desired, etc. The promoter is of special importance. In the context of the invention, it can be constitutive directing expression of the nucleic acid molecule in many types of host cells or specific to certain host cells (e.g. liver-specific regulatory sequences) or regulated in response to specific events or exogenous factors (e.g. by temperature, nutrient additive, hormone, etc.) or according to the phase of a viral cycle (e.g. late or early). One may also use promoters that are repressed during the production step in response to specific events or exogenous factors, in order to optimize virus production and circumvent potential toxicity of the expressed polypeptide(s).

Promoters suitable for constitutive expression in mammalian cells include but are not limited to the cytomegalovirus (CMV) immediate early promoter (U.S. Pat. No. 5,168,062), the RSV promoter, the adenovirus major late promoter, the phosphoglycerate kinase (PGK) promoter (Adra et al., 1987, Gene 60: 65-74), the thymidine kinase (TK) promoter of herpes simplex virus (HSV)-1 and the T7 polymerase promoter (WO98/10088). Vaccinia virus promoters are particularly adapted for expression in poxviruses. Representative examples include without limitation the vaccinia p7.5K, pH5R, p11K7.5 (Erbs et al., 2008, Cancer Gene Ther. 15(1): 18-28), pSE, pTK, p28, p11, pB2R, pF17R, pA14L, pSE/L, pA35R and pK1L promoters, as well as synthetic promoters such as those described in Chakrabarti et al. (1997, Biotechniques 23: 1094-7; Hammond et al, 1997, J. Virol Methods 66: 135-8; and Kumar and Boyle, 1990, Virology 179: 151-8) as well as early/late chimeric promoters. Promoters suitable for oncolytic measles viruses include without limitation any promoter directing expression of measles transcription units (Brandler and Tangy, 2008, CIMID 31: 271). Appropriate promoters for expression can be tested in vitro (e.g. in a suitable cultured cell line) or in vivo (e.g. in a suitable animal model or in the subject).

Those skilled in the art will appreciate that the regulatory elements controlling the expression of the nucleic acid molecule(s) inserted into the viral genome may further comprise additional elements for proper initiation, regulation and/or termination of transcription (e.g. polyA transcription termination sequences), mRNA transport (e.g. nuclear localization signal sequences), processing (e.g. splicing signals), and stability (e.g. introns and non-coding 5′ and 3′ sequences), translation (e.g. an initiator Met, tripartite leader sequences, IRES ribosome binding sites, signal peptides, etc.).

When appropriate, it may be advantageous to include additional regulatory elements to facilitate expression, trafficking and biological activity of at least one of gene inserted into the viral genome of the virus of the invention (i.e. SPD-TNFSF fusion protein).

An approach that may be pursued in the context of the present invention is coupling of the gene product encoded by the virus of the invention to an external agent such as a cytotoxic agent and/or a labelling agent. As used herein, the term “cytotoxic agent” refers to a compound that is directly toxic to cells, preventing their reproduction or growth such as toxins (e.g. an enzymatically active toxin of bacterial, fungal, plant or animal origin, or fragments thereof). As used herein, “a labelling agent” refers to a detectable compound. The labelling agent may be detectable by itself (e.g., radioactive isotope labels or fluorescent labels) or, in the case of an enzymatic label, may catalyze chemical modification of a substrate compound which is detectable. The coupling may be performed by genetic fusion between the gene product (SPD-TNFSF) and the external agent.

In a preferred embodiment, the oncolytic virus of the invention is a vaccinia virus (preferably from the Copenhagen strain) defective for both TK and RR activities (e.g. resulting from inactivating mutations in both the viral J2R and I4L genes) in the genome of which is inserted a nucleic acid molecule encoding a SPD-TNFSF fusion protein. Desirably, the elements are respectively placed under the transcriptional control of the pH5R promoter. Preferably, the SPD-TNFSF fusion protein encoding nucleic acid molecule is inserted within J2R (TK) locus of the viral genome.

In another preferred embodiment, the oncolytic virus of the invention is a vaccinia virus (preferably from the Copenhagen strain) defective for TK, RR and M2L activities (e.g. resulting from inactivating mutations in the viral J2R, I4L and M2L genes) in the genome of which is inserted a nucleic acid molecule encoding a SPD-TNFSF fusion protein. Desirably, the elements are respectively placed under the transcriptional control of the pH5R promoter. Preferably, the SPD-TNFSF fusion protein encoding nucleic acid molecule is inserted within J2R (TK) locus of the viral genome.

In an alternative, the oncolytic virus of the invention is a vaccinia virus (preferably from the Wyeth strain) defective for TK activity (resulting from inactivating mutations in the virus J2R gene) in the genome of which is inserted a nucleic acid molecule encoding a SPD-TNFSF fusion protein.

### Additional Therapeutic Polypeptide/Gene

While the virus according to the invention may encode only the SPD-TNFSF fusion protein of the invention (as defined above), it may also further encode another nucleic acid molecule encoding a polypeptide of interest.

Said another nucleic acid molecule encoding a polypeptide of interest is preferably a foreign nucleic acid (also called recombinant gene, transgene or nucleic acid).

In the context of the invention, the “foreign nucleic acid” that is inserted in the virus genome is not found in or expressed by a naturally occurring virus genome. Nevertheless, the foreign nucleic acid can be homologous or heterologous to the subject into which the recombinant virus is introduced. More specifically, it can be of human origin or not (e.g. of bacterial, yeast or viral origin except poxviral). Advantageously, said recombinant nucleic acid encodes a polypeptide or is a nucleic acid sequence capable of binding at least partially (by hybridization) to a complementary cellular nucleic acid (e.g., DNA, RNA, miRNA) present in a diseased cell with the aim of inhibiting a gene involved in said disease. Such a recombinant nucleic acid may be a native gene or portion(s) thereof (e.g. cDNA), or any variant thereof obtained by mutation, deletion, substitution and/or addition of one or more nucleotides.

In an advantageous embodiment, said a polypeptide of interest is therapeutic polypeptide. Accordingly, in this advantageous embodiment, the virus according to invention (as defined above), further comprises another nucleic acid molecule inserted in its genome encoding a therapeutic polypeptide.

By a “therapeutic polypeptide”, it is meant a polypeptide which is of therapeutic or prophylactic interest when administered appropriately to a subject, leading to a beneficial effect on the course or a symptom of the pathological condition to be treated or prevented.

The therapeutic polypeptide is preferably selected from the group consisting of an immunomodulatory polypeptide (preferably an immunostimulatory polypeptide), an antigenic polypeptide, a suicide gene product, an antibody, a functional derivative of an antibody, a functional fragment of an antibody, and any combination thereof.

In a preferred embodiment, the therapeutic polypeptide is an immunostimulatory polypeptide, preferably selected from the group consisting of cytokines, such as interleukins, chemokines, interferons, tumor necrosis factors, colony-stimulating factors; APC-exposed proteins; agonists of stimulatory immune checkpoints; antagonists of inhibitory immune checkpoints; and any combination thereof.

### Immunomodulatory Polypeptide

The term “immunomodulatory polypeptide” refers to a polypeptide targeting a component of a signalling pathway that can be involved in modulating an immune response either directly or indirectly. “Modulating” an immune response refers to any alteration in a cell of the immune system or in the activity of such a cell (e.g., a T cell). Such modulation includes stimulation or suppression of the immune system which can be manifested by an increase or decrease in the number of various cell types, an increase or decrease in the activity of these cells, or any other changes which can occur within the immune system. Preferably, such a polypeptide is capable of down-regulating at least partially an inhibitory pathway (antagonist) and/or of up-regulating at least partially a stimulatory pathway (agonist); in particular the immune pathway existing between an antigen presenting cell (APC) or a cancer cell and an effector T cell.

The immunomodulatory polypeptide that may be expressed by the vector according to the invention may act at any step of the T cell-mediated immunity including clonal selection of antigen-specific cells, T cell activation, proliferation, trafficking to sites of antigen and inflammation, execution of direct effector function and signalling through cytokines and membrane ligands. Each of these steps is regulated by counterbalancing stimulatory and inhibitory signals that in fine tune the response.

Suitable immunomodulatory polypeptides and methods of using them are described in the literature. Exemplary immunomodulatory polypeptides include, without limitation:
cytokines, such as interleukins, chemokines, interferons, tumor necrosis factors, colony-stimulating factors;
APC-exposed proteins;
agonists of a stimulatory immune checkpoint;
antagonists of an inhibitory immune checkpoint different from PD-L1; and
any combination thereof.

In one embodiment, the immunomodulatory polypeptide to be expressed by the vector according to the invention is a cytokine, preferably selected from the group consisting of;
interleukins (e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-36), IFNa, IFNg and granulocyte macrophage colony stimulating factor (GM-CSF);
chemokines (e.g. MIPIα, IL-8, CCL5, CCL17, CCL20, CCL22, CXCL9, CXCL10, CXCL11, CXCL13, CXCL12, CCL2, CCL19 and CCL21),
interferons (e.g. IFNα, IFNγ),
tumor necrosis factors (e.g. TNFα), and
colony-stimulating factors (e.g. granulocyte macrophage colony stimulating factor (GM-CSF)).

When the immunostimulatory polypeptide is a cytokine, it is preferably an interleukin or a colony-stimulating factor, with a specific preference for GM-CSF.

In another embodiment, the immunomodulatory polypeptide to be expressed by the vector according to the invention is an agonist of a stimulatory immune checkpoint or an antagonist of an inhibitory immune checkpoint.

The term “immune checkpoint” refers to a protein directly or indirectly involved in an immune pathway that under normal physiological conditions is crucial for preventing uncontrolled immune reactions and thus for the maintenance of self-tolerance and/or tissue protection. Immune checkpoints may be classified into two distinct categories: stimulatory and inhibitory immune checkpoints, respectively. A “stimulatory immune checkpoint” refers to an immune checkpoint involved in up-regulation of immune responses, while an “inhibitory immune checkpoint” is involved in down-regulation of immune responses.

Stimulatory immune checkpoints include CD28, ICOS, CD137 (4-1BB), OX40, CD70, CD40, and GITR, and the agonist of a stimulatory immune checkpoint is preferably selected from human ICOSL, 4-1BBL, OX40L, CD70, CD40L, GITRL and agonist antibodies to human ICOS (e.g. WO2018/187613), CD137 (4-1BB) (e.g. WO2005/035584), OX40 (e.g. U.S. Pat. No. 7,291,331 and WO03/106498), CD70 (e.g. WO2012/004367), CD40 (e.g. WO2017/184619), or GITR (e.g. WO2017/068186). As some agonists of stimulatory immune checkpoints are TNFSF members, when such an agonist of stimulatory immune checkpoints is further encoded by the vector according to the invention, it is preferably different from the member of the TNFSF or or receptor binding domain thereof of the fusion protein according to the invention.

Inhibitory immune checkpoints include PD-1, SIRPa, CD47, PD-L2, LAG3, Tim3, BTLA, and CTLA4, and the antagonist of an inhibitory immune checkpoint is preferably selected from antagonist antibodies human:
PD-1 (e.g. those described in WO2004/004771; WO2004/056875; WO2006/121168; WO2008/156712; WO2009/014708; WO2009/114335; WO2013/043569; and WO2014/047350, in particular nivolumab, pembrolizumab and cemiplimab),
SIRPa (e.g. WO2019/023347),
CD47 (e.g. WO2020/019135),
PD-L2 (e.g. WO2019/158645),
LAG3 (e.g. WO2018/071500),
Tim3, (e.g. WO2020/093023)
BTLA (e.g. WO2010/106051), and
CTLA4 (e.g. those described in U.S. Pat. No. 8,491,895, WO2000/037504, WO2007/113648, WO2012/122444 and WO2016/196237 among others, and in particular ipilimumab marketed by Bristol Myer Squibb as Yervoy® (see e.g. U.S. Pat. Nos. 6,984,720; 8,017,114), MK-1308 (Merck), AGEN-1884 (Agenus Inc.; WO2016/196237) and tremelimumab (AstraZeneca; U.S. Pat. Nos. 7,109,003 and 8,143,379) and single chain anti-CTLA4 antibodies (see e.g. WO97/20574 and WO2007/123737).

### Antigenic Polypeptide

The term “antigenic” refers to the ability to induce or stimulate a measurable immune response in a subject into which the virus of the invention (as described herein) encoding the polypeptide qualified as antigenic has been introduced. The stimulated or induced immune response against the antigenic polypeptide expressed by said virus can be humoral and/or cellular (e.g. production of antibodies, cytokines and/or chemokines involved in the activation of effector immune cells). The stimulated or induced immune response usually contributes in a protective effect in the administered subject. A vast variety of direct or indirect biological assays are available in the art to evaluate the antigenic nature of a polypeptide either in vivo (animal or human subjects), or in vitro (e.g. in a biological sample). For example, the ability of a particular antigen to stimulate innate immunity can be performed by for example measurement of the NK/NKT-cells (e.g. representativity and level of activation), as well as, IFN-related cytokine and/or chemokine producing cascades, activation of TLRs (for Toll-like receptor) and other markers of innate immunity (Scott-Algara et al., 2010 PLOS One 5(1), e8761; Zhou et al., 2006, Blood 107, 2461-2469; Chan, 2008, Eur. J. Immunol. 38, 2964-2968). The ability of a particular antigen to stimulate a cell-mediated immune response can be performed for example by quantification of cytokine(s) produced by activated T cells including those derived from CD4+ and CD8+ T-cells using routine bioassays (e.g. characterization and/or quantification of T cells by ELISpot, by multiparameters flow cytometry, ICS (for intracellular cytokine staining), by cytokine profile analysis using multiplex technologies or ELISA), by determination of the proliferative capacity of T cells (e.g. T cell proliferation assays by [3H] thymidine incorporation assay), by assaying cytotoxic capacity for antigen-specific T lymphocytes in a sensitized subject or by identifying lymphocyte subpopulations by flow cytometry and by immunization of appropriate animal models, as described herein.

It is contemplated that the term antigenic polypeptide encompasses native antigen as well as fragment (e.g. epitopes, immunogenic domains, etc.) and variant thereof, provided that such fragment or variant is capable of being the target of an immune response. Preferred antigenic polypeptides for use herein are tumor-associated antigens. It is within the scope of the skilled artisan to select the one or more antigenic polypeptide that is appropriate for treating a particular pathological condition.

In one embodiment, the antigenic polypeptide(s) encoded by the recombinant modified virus is/are cancer antigen(s) (also called tumor-associated antigens or TAA) that is associated with and/or serve as markers for cancers. Cancer antigens encompass various categories of polypeptides, e.g. those which are normally silent (i.e. not expressed) in healthy cells, those that are expressed only at low levels or at certain stages of differentiation and those that are temporally expressed such as embryonic and foetal antigens as well as those resulting from mutation of cellular genes, such as oncogenes (e.g. activated ras oncogene), proto-oncogenes (e.g. ErbB family), or proteins resulting from chromosomal translocations.

Numerous tumor-associated antigens are known in the art. Exemplary tumor antigens include without limitation, colorectal associated antigen (CRC), Carcinoembryonic Antigen (CEA), Prostate Specific Antigen (PSA), BAGE, GAGE or MAGE antigen family, p53, mucin antigens (e.g. MUC1), HER2/neu, p21ras, hTERT, Hsp70, iNOS, tyrosine kinase, mesothelin, c-erbB-2, alpha fetoprotein, AM-1, among many others, and any immunogenic epitope or variant thereof.

The tumor-associated antigens may also encompass neo-epitopes/antigens that have emerged during the carcinogenesis process in a cancer cell and comprising one or more mutation(s) of amino acid residue(s) with respect to a corresponding wild-type antigen. Typically, it is found in cancer cells or tissues obtained from a patient but not found in a sample of normal cells or tissues obtained from a patient or a heathy individual.

The tumor-associated antigens may also encompass antigens encoded by pathogenic organisms that are capable of inducing a malignant condition in a subject (especially chronically infected subject) such as RNA and DNA tumor viruses (e.g. human papillomavirus (HPV), hepatitis C virus (HCV), hepatitis B virus (HBV), Epstein Barr virus (EBV), etc.) and bacteria (e.g. Helicobacter pylori).

In another embodiment, the antigenic polypeptide(s) encoded by the virus of the invention is/are vaccinal antigen(s) that, when delivered to a human or animals subject, aim(s) at protecting therapeutically or prophylactically against infectious diseases. Numerous vaccine antigens are known in the art. Exemplary vaccine antigens include but are not limited to cellular antigens, viral, bacterial or parasitic antigens. Cellular antigens include the mucin 1 (MUC1) glycoprotein. Viral antigens include for example antigens from hepatitis viruses A, B, C, D and E, immunodeficiency viruses (e.g. HIV), herpes viruses, cytomegalovirus, varicella zoster, papilloma viruses, Epstein Barr virus, influenza viruses, para-influenza viruses, coxsakie viruses, picorna viruses, rotaviruses, respiratory syncytial viruses, rhinoviruses, rubella virus, papovirus, mumps virus, measles virus and rabies virus. Some non-limiting examples of HIV antigens include gp120 gp40, gp160, p24, gag, pol, env, vif, vpr, vpu, tat, rev, nef tat, nef. Some non-limiting examples of human herpes virus antigens include gH, gL gM gB gC gK gE or gD or Immediate Early protein such aslCP27, ICP47, ICP4, ICP36 from HSV1 or HSV2. Some non-limiting examples of cytomegalovirus antigens include gB. Some non-limiting examples of derived from Epstein Barr virus (EBV) include gp350. Some non-limiting examples of Varicella Zoster Virus antigens include gp1, 11, 111 and IE63. Some non-limiting examples of hepatitis C virus antigens includes env E1 or E2 protein, core protein, NS2, NS3, NS4a, NS4b, NS5a, NS5b, p7. Some non-limiting examples of human papilloma viruses (HPV) antigens include L1, L2, E1, E2, E3, E4, E5, E6, E7. Antigens derived from other viral pathogens, such as Respiratory Syncytial virus (e.g. F and G proteins), parainfluenza virus, measles virus, mumps virus, flaviviruses (e.g. Yellow Fever Virus, Dengue Virus, Tick-borne encephalitis virus, Japanese Encephalitis Virus) and Influenza virus cells (e.g. HA, NP, NA, or M proteins) can also be used in accordance with the present invention. Bacterial antigens include for example antigens from Mycobacteria causing TB, leprosy, pneumococci, aerobic gram negative bacilli, mycoplasma, staphylococcus, streptococcus, salmonellae, chlamydiae, neisseriae and the like. Parasitic antigenic polypeptides include for example antigens from malaria, leishmaniasis, trypanosomiasis, toxoplasmosis, schistosomiasis and filariasis.

### Antibodies and Antigen-Binding Fragments or Derivatives Thereof

Any antibody or antigen-binding fragment or derivative thereof with therapeutic activity may further be encoded by the virus of the invention, including anti-neoplastic antibodies or antigen-binding fragments or derivatives thereof, in particular antibodies or antigen-binding fragments or derivatives thereof that affect the regulation of cell surface receptors, such as anti HER2 antibodies (e.g. trastuzumab), anti-EGFR antibodies (e.g. cetuximab, panitumumab, zalutumumab, nimotuzumab, matuzumab), anti-VEGF antibodies (e.g. bevacizumab and ranibizumab) or antigen-binding fragments or derivatives thereof.

In the context of the invention, “antibody” (“Ab”) is used in the broadest sense and is preferably as defined in the section “General definitions” above.

The antibody is preferably a monoclonal antibody, preferably humanized or chimeric.

Representative examples of antigen-binding fragments are known in the art, including Fab, Fab′, F(ab′)2, dAb, Fd, Fv, scFv, ds-scFv and diabody. A particularly useful antibody fragment is a single chain antibody (scFv) comprising the two domains of a Fv fragment, VL and VH, that are fused together, eventually with a linker to make a single protein chain.

### Production of a SPD-TNFSF Fusion Protein

In one embodiment, the virus may also be utilized in the context of the invention for producing by recombinant means the one or more SPD-TNFSF fusion protein that it encodes. It may advantageously comprise one or more additional element(s) enabling maintenance, propagation or expression of the nucleic acid molecule encoding the SPD-TNFSF fusion protein in a host cell. Such additional elements comprise marker gene(s) in order to facilitate identification and isolation of the producer host cells (e.g. by complementation of a cell auxotrophy or by antibiotic resistance). Suitable marker genes include without limitation dihydrofolate reductase (dhfr) which confers resistance to methotrexate (Wigler et al., 1980, Proc. Natl. Acad. Sci. USA 77: 3567; O'Hare et al., 1981, Proc. Natl. Acad. Sci. USA 78: 1527); gpt which confers resistance to mycophenolic acid (Mulligan and Berg, 1981, Proc. Natl. Acad. Sci. USA 78: 2072); neo which confers resistance to the aminoglycoside G-418 (Colberre-Garapin et al., 1981, J. Mol. Biol. 150: 1); zeo which confers resistance to zeomycin, kana which confers resistance to kanamycin; hygro, which confers resistance to hygromycin (Santerre et al., 1984, Gene 30: 147). Recombinant viruses lacking a functional TK (e.g. resulting from insertion of the nucleic acid molecule encoding the SPD-TNFSF protein into the J2R (TK) locus) may be selected with media containing bromodeoxyuridine (BrdU). Indeed, TK− viruses are insensitive to the BrdU drug whereas the drug interferes with DNA synthesis in TK+ viruses. One may also rely on reporter luminescent or colorimetric systems, e.g. based on GFP (green fluorescent protein), luciferase and beta-galactosidase.

The methods for recombinantly producing the SPD-TNFSF fusion protein are conventional in the art. Typically such methods comprise (a) introducing the virus described herein into a suitable producer cell to produce a transfected or infected producer cell, (b) culturing in-vitro said transfected or infected producer cell under conditions suitable for its growth, (c) recovering the one or more SPD-TNFSF fusion protein(s) from the cell culture, and (d) optionally, purifying the recovered SPD-TNFSF fusion protein(s). In the context of the invention, producer cells are preferably human or non-human eukaryotic cells. Preferred producer cells include without limitation hamster cell lines such as BHK-21 (ATCC CCL-10), CV-1 (African monkey kidney cell line), COS (e.g. COS-7) cells, Chinese hamster ovary (CHO) cells, mouse NIH/3T3 cells, mouse NSO myeloma cells, human cell lines such as HeLa (ATCC-CRM-CCL-2™ or ATCC-CCL-2.2™), Vero cells, HEK293 cells (Graham et al., 1997, J. Gen. Virol. 36: 59-72) HER96 and PERC.6 cells (Fallaux et al., 1998, Human Gene Ther. 9: 1909-17), avian cells (e.g. chicken, duck cells as described herein and described in WO2005/042728, WO2006/108846, WO2008/129058, WO2010/130756, WO2012/001075 etc.) as well as primary chicken embryo fibroblasts (CEF) prepared from chicken embryos obtained from fertilized eggs.

The producer cells can be cultured in conventional fermentation bioreactors, flasks, and petri plates. Culturing can be carried out at a temperature, pH and oxygen content appropriate for a given host cell. No attempts to describe in detail the various methods known to produce proteins in eukaryotic cells will be made here. Production of the SPD-TNFSF fusion protein can be intracellular or preferably secreted outside the producer cell (e.g. in the culture medium).

The virus can be at least partially isolated before being used according to the present invention. Various purification steps can be envisaged, including clarification, enzymatic treatment (e.g. benzonase, protease), chromatographic and filtration steps. Appropriate methods are described in the art (e.g. WO2007/147528; WO2008/138533, WO2009/100521, WO2010/130753, WO2013/022764).

The SPD-TNFSF fusion protein can then be purified by well-known purification methods. The conditions and technology used to purify a particular protein will depend on factors such as the expression conditions, net charge, molecular weight, hydrophobicity, hydrophilicity and will be apparent to those having skill in the art. Moreover, the level of purification will depend on the intended use. If necessary, especially when the SPD-TNFSF fusion protein is not secreted outside the producer cell or where it is not secreted completely, it can be recovered by standard lysis procedures, including freeze thaw, sonication, mechanical disruption, use of lysing agents and the like. If secreted, it can be recovered directly from the culture medium. Various purification steps can be envisaged, including without limitation clarification (e.g. ammonium sulfate precipitation, acid extraction), enzymatic treatment (e.g. benzonase, protease), chromatographic (e.g. reverse phase, size exclusion, ion exchange, affinity, phosphocellulose, hydrophobic-interaction or hydroxyapatite chromatography, etc.) and filtration steps. Appropriate methods are described in the art (e.g. WO2007/147528; WO2008/138533, WO2009/100521, WO2010/130753, WO2013/022764). Desirably, the SPD-TNFSF fusion protein recombinantly produced from the virus of the invention is at least partially purified in the sense that it is substantially free of other cellular material. Further, the SPD-TNFSF fusion protein may be formulated according to the conditions conventionally used in the art (e.g. WO2009/073569).

### Therapeutic Use

The present invention also provides a composition comprising a therapeutically effective amount of the SPD-TNFSF fusion protein, the virus of the invention, optionally with a pharmaceutically acceptable vehicle. Such a composition may be administered once or several times and via the same or different routes.

A “therapeutically effective amount” corresponds to the amount of SPD-TNFSF fusion protein or virus that is sufficient for producing one or more beneficial results. Such a therapeutically effective amount may vary as a function of various parameters, in particular the mode of administration; the disease state; the age and weight of the subject; the ability of the subject to respond to the treatment; kind of concurrent treatment; the frequency of treatment; and/or the need for prevention or therapy. When prophylactic use is concerned, the SPD-TNFSF fusion protein, the virus or the composition of the invention is administered at a dose sufficient to prevent or to delay the onset and/or establishment and/or relapse of a pathologic condition (e.g. a proliferative disease such as cancer), especially in a subject at risk. For “therapeutic” use, the SPD-TNFSF fusion protein, the virus or the composition of the present invention is administered to a subject diagnosed as having a pathological condition (e.g. a proliferative disease such as cancer or disorders associated with TNF cytokine dysfunction) with the goal of treating the disease, eventually in association with one or more conventional therapeutic modalities. In particular, a therapeutically effective amount could be that amount necessary to cause an observable improvement of the clinical status over the baseline status or over the expected status if not treated, e.g. stabilization (i.e. not worsening) of the state of disease, delay or slowing of disease progression or severity, amelioration or palliation of the disease state, prolonged survival, better response to the standard treatment, improvement of quality of life, reduced mortality, reduction in the tumor number, reduction in the tumor size, reduction in the number or extend of metastasis, increase in the length of remission, etc. A therapeutically effective amount could also be the amount necessary to cause the development of an effective non-specific (innate) and/or specific immune response such as tumor immune response. Typically, development of an immune response in particular T cell response can be evaluated in vitro, in suitable animal models or using biological samples collected from the subject. For example, techniques routinely used in laboratories (e.g. flow cytometry, histology) may be used to perform tumor surveillance. One may also use various available antibodies so as to identify different immune cell populations involved in anti-tumor response that are present in the treated subjects, such as cytotoxic T cells, activated cytotoxic T cells, natural killer cells and activated natural killer cells. An improvement of the clinical status can be easily assessed by any relevant clinical measurement typically used by physicians or other skilled healthcare staff.

The term “pharmaceutically acceptable vehicle” is intended to include any and all carriers, solvents, diluents, excipients, adjuvants, dispersion media, coatings, antibacterial and antifungal agents, absorption agents and the like compatible with administration in mammals and in particular human subjects.

The SPD-TNFSF fusion protein, the virus or the composition thereof can be placed in a solvent or diluent appropriate for human or animal use. The solvent or diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see for example the most current edition of Remington: The Science and Practice of Pharmacy, A. Gennaro, Lippincott, Williams&Wilkins).

In one embodiment, the SPD-TNFSF fusion protein, the virus or the composition thereof is suitably buffered for human use. Suitable buffers include without limitation phosphate buffer (e.g. PBS), bicarbonate buffer and/or Tris buffer capable of maintaining a physiological or slightly basic pH (e.g. from approximately pH 7 to approximately pH 9).

The SPD-TNFSF fusion protein, the virus or the composition thereof may also contain other pharmaceutically acceptable excipients for providing desirable pharmaceutical or pharmacodynamic properties, including for example osmolarity, viscosity, clarity, colour, sterility, stability, rate of dissolution of the formulation, modifying or maintaining release or absorption into the human or animal subject, promoting transport across the blood barrier or penetration in a particular organ.

In one embodiment, the virus compositions can also comprise one or more adjuvant(s) capable of stimulating immunity (especially a T cell-mediated immunity) or facilitating infection of tumor cells upon administration, e.g. through toll-like receptors (TLR) such as TLR-7, TLR-8 and TLR-9, including without limitation alum, mineral oil emulsion such as, Freunds complete and incomplete (IFA), lipopolysaccharide or a derivative thereof (Ribi et al., 1986, Immunology and Immunopharmacology of Bacterial Endotoxins, Plenum Publ. Corp., NY, p407-419), saponins such as QS21 (Sumino et al., 1998, J. Virol. 72: 4931; WO98/56415), imidazoquinoline compounds such as Imiquimod (Suader, 2000, J. Am Acad Dermatol. 43:S6), S-27609 (Smorlesi, 2005, Gene Ther. 12: 1324) and related compounds such as those described in WO2007/147529, cytosine phosphate guanosine oligodeoxynucleotides such as CpG (Chu et al., 1997, J. Exp. Med. 186: 1623; Tritel et al., 2003, J. Immunol. 171: 2358) and cationic peptides such as IC-31 (Kritsch et al., 2005, J. Chromatogr Anal. Technol. Biomed. Life Sci. 822: 263-70).

In one embodiment, the SPD-TNFSF fusion protein, the virus or the composition of the present invention may be formulated with the goal of improving its stability in particular under the conditions of manufacture and long-term storage (i.e. for at least 6 months, with a preference for at least two years) at freezing (e.g. −70° C., −20° C.), refrigerated (e.g. 4° C.) or ambient temperatures. Various virus formulations are available in the art either in frozen, liquid form or lyophilized form (e.g. WO98/02522, WO01/66137, WO03/053463, WO2007/056847 and WO2008/114021, WO2016087457, etc.). Solid (e.g. dry powdered or lyophilized) compositions can be obtained by a process involving vacuum drying and freeze-drying (see e.g. WO2014/053571). For illustrative purposes, buffered formulations including NaCl and/or sugar are particularly adapted to the preservation of viruses (e.g. S01 buffer: 342.3 g/L saccharose, 10 mM Tris, 1 mM MgCl₂, 150 mM NaCl, 54 mg/L, Tween 80; ARME buffer: 20 mM Tris, 25 mM NaCl, 2.5% Glycerol (w/v), pH 8.0; S520 buffer: 100 g/L saccharose, 30 mM Tris, pH 7.6; S08 buffer: 10 mM Tris, 50 mM NaCl, 50 g/L saccharose, 10 mM Sodium glutamate, pH 8.0).

In certain embodiments, the virus composition of the present invention can be formulated to ensure proper distribution or a delayed release in vivo. For example, it can be formulated in liposomes. Biodegradable, biocompatible polymers can be used, such as ethylene vinyl acetate, polyanhydrides, polyglycolic acid, collagen, polyorthoester, and polylactic acid. Many methods for the preparation of such formulations are described by e.g. J. R. Robinson in “Sustained and Controlled Release Drug Delivery Systems”, ed., Marcel Dekker, Inc., New York, 1978.

The appropriate dosage of SPD-TNFSF fusion protein can be adapted as a function of various parameters and may be routinely determined by a practitioner in the light of relevant circumstances. Suitable dosage for the SPD-TNFSF fusion protein varies from 0.001 to approximately 100 mg/kg depending on the route of administration protein and the quantitative technique used. As a general guidance, the quantity of SPD-TNFSF fusion protein present in a sample can be determined by routine titration techniques, e.g. UV absorbance or ELISA.

The appropriate dosage of virus can be adapted as a function of various parameters and may be routinely determined by a practitioner in the light of the relevant circumstances. Suitable dosage for the virus varies from approximately 10⁴ to approximately 10¹³ vp (viral particles), iu (infectious unit) or pfu (plaque-forming units) depending on the virus and the quantitative technique used. As a general guidance, vaccinia virus doses from approximately 10⁴ to approximately 10¹³ pfu are suitable, preferably from approximately 10⁶ pfu to approximately 10¹¹ pfu, more preferably from approximately 10⁷ pfu to approximately 5×10⁹ pfu; doses of approximately 10⁸ pfu to approximately 10⁹ pfu being particularly preferred especially for human use. The quantity of virus present in a sample can be determined by routine titration techniques, e.g. by counting the number of plaques following infection of permissive cells using permissive cells (e.g. BHK-21 or CEF), immunostaining (e.g. using anti-virus antibodies; Caroll et al., 1997, Virology 238: 198-211), by measuring the A260 absorbance (vp titers), or still by quantitative immunofluorescence (iu titers).

### Administration

The SPD-TNFSF fusion protein, the virus or the composition of the present invention may be administered in a single dose (e.g. bolus injection) or multiple doses. If multiple administrations, they may be performed by the same or different routes and may take place at the same site or at alternative sites. It is also possible to proceed via sequential cycles of administrations that are repeated after a rest period. Intervals between each administration can be from several hours to one year (e.g. 24 h, 48 h, 72 h, weekly, every two weeks, monthly or yearly). Intervals can also be irregular (e.g. following tumor progression). The doses can vary for each administration within the range described above.

Any of the conventional administration routes are applicable in the context of the invention including parenteral, topical or mucosal routes. Parenteral routes are intended for administration as an injection or infusion. Common parenteral injection types are intravenous (into a vein), intra-arterial (into an artery), intradermal (into the dermis), subcutaneous (under the skin), intramuscular (into muscle) and intratumoral (into tumor or at its close proximity). Infusions typically are given by intravenous route. Mucosal administrations include without limitation oral/alimentary, intranasal, intratracheal, intrapulmonary, intravaginal or intra-rectal route. Topical administration can also be performed using transdermal means (e.g. patch and the like). Administrations may use conventional syringes and needles (e.g. Quadrafuse injection needles) or any compound or device available in the art capable of facilitating or improving delivery of the active agent(s) in the subject. Preferred routes of administration for the virus include intravenous and intratumoral routes.

In the context of the invention, the virus may be administered once or several time (e.g. 2, 3, 4, 5, 6, 7 or 8 times etc.) at a dose within the range of from 10⁷ to 5×10⁹ pfu. The time interval between each administration can vary from approximately 1 day to approximately 8 weeks, advantageously from approximately 2 days to approximately 6 weeks, preferably from approximately 3 days to approximately 4 weeks and even more preferably from approximately 1 week to approximately 3 weeks (e.g. every two weeks for example). A preferred therapeutic scheme involves from 2 to 5 (e.g. 3) intravenous or intratumoral administrations of 10⁸ or 10⁹ pfu of oncolytic vaccinia virus at approximately 1 or 2 weeks interval.

The present invention also relates to a method for treating a proliferative disease such as cancer or disorders associated with TNF cytokine dysfunction such as infectious disease, inflammatory diseases, metabolic diseases, autoimmune diseases, degenerative diseases, apoptosis-associated diseases, and transplant rejections comprising administering a virus as described herein to a subject in need thereof.

In one embodiment, the present invention also relates to a method for treating a proliferative disease such as cancer comprising administering a virus as described herein to a subject in need thereof.

In one embodiment, the present invention also relates to a method for inhibiting tumor cell growth in vivo comprising administering a virus as described herein to a subject in need thereof.

In one embodiment, the present invention also relates to a method for enhancing in immune response to tumor cells comprising administering a virus as described herein to a subject in need thereof.

In one embodiment, the administration of the virus for use in the present invention elicits, stimulates and/or re-orients an immune response. In particular, the administration induces a protective T or B cell response in the treated host, e.g. against said virus or eventually against the product encoded by the SPD-TNFSF nucleic acid molecule(s) inserted in the viral genome if any. The protective T response can be CD4+ or CD8+ or both CD4+ and CD8+ cell mediated. B cell response can be measured by ELISA and T cell response can be evaluated by conventional ELISpot, ICS assays from any sample (e.g. blood, organs, tumors, etc.) collected from the immunized animal or subject.

In one embodiment, the administration of the oncolytic virus also permits to change tumor microenvironment with the goal of enhancing activity of effector cells in the tumor, especially effector T lymphocytes and/or promoting at least partial Treg depletion. Tumor infiltrating cells can be easily identified for examples by conventional immunostaining assays.

A higher therapeutic efficacy could be evidenced as described above in connection with the term “therapeutically effective amount” with a specific preference for a longer survival.

Examples of disorders that may be treated using the SPD-TNFSF fusion protein, virus, composition, or methods of the invention include without limitation:
Proliferative diseases
   Cancer such as, e.g. bone cancer, liver cancer, pancreatic cancer, stomach cancer, colon cancer, cancer of the esophagus, oropharyngeal cancer, lung cancer, head and neck cancer, skin cancer, melanoma, uterine cancer, endometrial cancer, cervix cancer, ovarian cancer, breast cancer, rectal cancer, cancer of the anal region, prostate cancer, lymphoma, cancer of the endocrine system, cancer of the thyroid gland, sarcoma of soft tissue, chronic or acute leukemias, bladder cancer, renal cancer, neoplasm of the central nervous system (CNS), glioma, glioblastoma, etc. Preferred cancer that may be treated using the SPD-TNFSF fusion protein or the virus of the invention include cancer typically responsive to immunotherapy. Non-limiting examples of preferred cancer for treatment include melanoma (e.g. metastatic malignant melanoma), renal cancer (e.g. clear cell carcinoma), prostate cancer (e.g. hormone refractory prostate adenocarcinoma), breast cancer, colorectal cancer, lung cancer (e.g. non-small cell lung cancer) and liver cancer (e.g. hepatocarcinoma).
   Cardiovascular diseases such as e.g. restenosis
Disorders associated with dysfunction of TNF cytokines:
   Infectious disease such as, e.g. HIV infection, particularly chronic viral diseases, such as hepatitis A, B or C, herpes, tuberculosis, Epstein-Barr virus, cytomegalovirus, John Cunningham virus and human papilloma virus, yellow fever, dengue, flaviviruses, influenza viruses, hemorrhagic infectious diseases (Marburg or Ebola viruses), and severe acute respiratory syndrome (SARS), bacterial infectious diseases, such as Legionnaire's disease (Legionella), sexually transmitted diseases (e.g. chlamydia or gonorrhea), gastric ulcer (Helicobacter), cholera (vibrio), diphtheria, infections by E. coli, Staphylococci, Salmonella or Streptococci (tetanus); infections by protozoan pathogens such as malaria, sleeping sickness, leishmaniasis; toxoplasmosis, i.e. infections by Plasmodium, Trypanosoma, Leishmania and Toxoplasma; or fungal infections, which are caused, e.g. by Cryptococcus neoformans, Histoplasma capsulatum, Coccidioides immitis, Blastomyces dermatitidis or Candida albicans;
   Inflammatory disease such as, e.g. celiac disease, vasculitis, lupus, chronic obstructive pulmonary disease (COPD), irritable bowel disease, atherosclerosis, arthritis, ankylosing spondylitis, Crohn's disease, colitis, chronic active hepatitis, dermatitis and psoriasis;
   Metabolic disease such as, e.g. diabetes, cystinosis, dyslipidemia hyperthyroidism, hypothyroidism, hyperlipidemia, hypolipidomia, galactosemia, obesity, Gaucher's disease and phenylketonuria;
   Autoimmune disease such as, e.g. systemic lupus erythematosus, rheumatoid arthritis and Sjögren's syndrome
   Degenerative disease e.g. neurodegenerative disease such as Alzheimer's disease, Parkinson's disease, amyotrophic lateral sclerosis (ALS), Huntington's disease, macular degeneration, multiple sclerosis, muscular dystrophy, Niemann Pick disease, neuronal ceroid lipofuscinosis, osteoporosis
   Apoptosis-associated diseases
   Transplant rejections

The SPD-TNFSF fusion protein, virus, composition or method according to the invention can be associated with one or more substances or therapy effective in anticancer therapy and the present invention also concerns a method which comprises the step of delivering to the subject an additional cancer therapy. In the context of the present invention, said additional cancer therapy comprises surgery, radiation, chemotherapy, immunotherapy, hormone therapy or a combination thereof. In a preferred embodiment the method of the invention comprises the administration of one or more substances effective in anticancer therapy. Among pharmaceutical substances effective in anticancer therapy which may be used in association or in combination with the SPD-TNFSF fusion protein, virus, composition, or method according to the invention, there may be mentioned more specifically:
alkylating agents such as e.g. mitomycin C, cyclophosphamide, busulfan, ifosfamide, isosfamide, melphalan, hexamethylmelamine, thiotepa, chlorambucil, or dacarbazine;
antimetabolites such as, e.g. gemcitabine, capecitabine, 5-fluorouracil, cytarabine, 2-fluorodeoxy cytidine, methotrexate, idatrexate, tomudex or trimetrexate;
topoisomerase II inhibitors such as, e.g. doxorubicin, epirubicin, etoposide, teniposide or mitoxantrone;
topoisomerase I inhibitors such as, e.g. irinotecan (CPT-11), 7-ethyl-10-hydroxy-camptothecin (SN-38) or topotecan;
antimitotic drugs such as, e.g., paclitaxel, docetaxel, vinblastine, vincristine or vinorelbine;
platinum derivatives such as, e.g., cisplatin, oxaliplatin, spiroplatinum or carboplatinum;
inhibitors of tyrosine kinase receptors such as sunitinib (Pfizer) and sorafenib (Bayer);
anti-neoplastic antibodies in particular antibodies that affect the regulation of cell surface receptors such as trastuzumab, cetuximab, panitumumab, zalutumumab, nimotuzumab, matuzumab, bevacizumab and ranibizumab.
EGFR (for Epidermal Growth Factor Receptor) inhibitors such as gefitinib, erlotinib and lapatinib; and
immunomodulatory agents such as, e.g. alpha, beta or gamma interferon, interleukin (in particular IL-2, IL-6, IL-10 or IL-12) or tumor necrosis factor;

The SPD-TNFSF fusion protein, expression vector, composition, or method according to the invention can also be used in association with radiotherapy.

The present invention also provides kits including a different container (e.g., a sterile glass or plastic vial) for each virus dose to be administered. Optionally, the kit can include a device for performing the administration of the active agents. The kit can also include a package insert including information concerning the compositions or individual component and dosage forms in the kit.

### Material and Methods

Those skilled in the art will recognize or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

### Viruses and Plasmids

VVTG18058 (empty VACV, VACV control, or unarmed control VACV) is a Vaccinia virus (Copenhagen strain) deleted of J2R and I4L genes. VVTG18058 was used as an unarmed control virus. VVTG18058 was produced on chicken embryo fibroblasts (CEF). Titration was performed by plaque assay on Vero cells.

The plasmid pTG19274 is a plasmid encoding for an irrelevant FLAG-tagged molecule. Plasmid pTG19274 is used as a negative control.

The plasmid pTG19333 is a plasmid without transgene. Plasmid pTG19333 is used as a negative control.

The plasmid pTG19325 is a plasmid encoding for the fusion protein SPD (M1-G257, P35247 numbering)-CD40L (H47 to L261 from P29965 numbering) construct (SEQ ID NO:50).

The plasmid pTG19344 is a plasmid encoding for a soluble CD40L (G116 to L261) consisting of a heterologous signal peptide followed by the CD40L extracellular domain (SEQ ID NO:51).

The plasmid pTG19965 is a plasmid encoding for a heterologous signal peptide upstream of human SPD (A21 to G257)-CD40L (N119 to L261). This construction (SEQ ID NO:52) contains a heterologous signal peptide, a shorter form of CD40L, and a FLAG tag in C-terminus.

The plasmid pTG20032 is a plasmid encoding for a 4-1-BBL (D80 to E254) ectodomain consisting of a heterologous signal peptide followed by the 4-1-BBL extracellular domain (SEQ ID NO:53).

Transfer plasmid carrying the different SPD-TNFSF constructions according to the invention are described in Table 2 below.

TABLE 2
Design of SPD-TNFSF constructs according to
the invention and corresponding plasmids.

| | SEQ ID NO of | |
|---|---|---|
| | constructs | |
| | encoded by the | SPD-TNFSF fusion |
| Plasmid ID | plasmid | protein structure |
| pTG19966 | 2 | [N-terminus]-[coiled-coil neck |
| | | domain]-[CD40L] |
| pTG19967 | 10 | [N-terminus]-[coiled-coil neck |
| | | domain]-[linker]-[CD40L] |
| pTG19968 | 3 | [N-terminus]-[collagen domain(12)]- |
| | | [coiled-coil neck domain]-[CD40L] |
| pTG19969 | 11 | [N-terminus]-[collagen domain(12)]- |
| | | [coiled-coil neck domain]- |
| | | [linker]-[CD40L] |
| pTG20038 | 5 | [N-terminus]-[collagen domain(6 + |
| | | N-Gly)]-[coiled-coil neck |
| | | domain]-[CD40L] |
| pTG20039 | 6 | [N-terminus]-[collagen domain(6 − |
| | | N-Gly)]-[coiled-coil neck |
| | | domain]-[CD40L] |
| pTG20041 | 4 | [N-terminus]-[collagen domain(12bis)]- |
| | | [coiled-coil neck domain]-[CD40L] |
| pTG20040 | 7 | [N-terminus]-[collagen domain(19)]- |
| | | [coiled-coil neck domain]-[CD40L] |
| pTG20042 | 8 | [N-terminus]-[collagen domain(30)]- |
| | | [coiled-coil neck domain]-[CD40L] |
| pTG20033 | 9 | [N-terminus]-[collagen domain(12)]- |
| | | [coiled-coil neck domain]-[4-1BBL] |

([N-terminus] defined by the SEQ ID NO:1, [coiled-coil neck domain] by the SEQ ID NO: 23, [CD40L] by the SEQ ID NO: 24, [4-1BBL] by the SEQ ID NO:28 and Collagen domains defined according to the table 1).
COPTG19967 is a recombinant Copenhagen vaccinia virus double deleted (tk- and rr-) wherein the expression sequence of pTG19967 has been inserted within its J2R locus.
COPTG19968 is a recombinant Copenhagen vaccinia virus double deleted (tk- and rr-) wherein the expression sequence of pTG19968 has been inserted within its J2R locus.
COPTG19969 is a recombinant Copenhagen vaccinia virus double deleted (tk- and rr-) wherein the expression sequence of pTG19969 has been inserted within its J2R locus.

### Infection/Transfection Experiments

A method for expression of a recombinant protein is the infection/transfection method. Such method consists of infecting a cell, for example a HeLa cell, with a vaccinia virus (a poxvirus) and transfecting said cell with a plasmid encoding the gene of interest under regulation control of a poxvirus promotor. Such method allows the expression of the encoded gene of interest within the cell. The expression product may be recovered within the supernatant for further analysis.

Co-infection/transfection in HeLa cells was carried out with the goal of selecting the most effective SPD-TNFSF constructions to be vectorized in VACV (vaccinia virus) genome.

Briefly, cells were seeded, two days prior infection, at 4E+05 cells/well/3 mL of complete medium (DMEM 4 Gibco ref. 41966-029; Glutamine 2 mM; Gentamicin 40 μg/mL; 10% fetal bovine serum (FBS)) in 6-well-plates.

Before infection, the culture media was removed and replaced by 400 μL of vaccinia virus preparation (VVTG18058) in PBS+ (PBS+1% cations) corresponding to a MOI 1. After 30 min. at room temperature (RT), the viral inoculum was removed and replaced by 1.2 mL of complete medium without FBS. The plates were incubated for 2 h at 37° C. with 5% CO₂. Transfection was then performed by addition of 1 μg of plasmid DNA formulated with 4.5 μL of Lipofectamine 2000 (Invitrogen, 11668-027) in each well, following the provider's protocols. pTG19274 (encoding irrelevant FLAG-tagged molecule) was used as a negative control. The infection/transfections were performed in triplicate. The plates were incubated 48 hours at 37° C. and 5% CO₂. The culture supernatants were then collected, centrifuged and filtrated on 0.1 μm filters to remove all virus particles and cellular debris. The clarified supernatants were stored at −80° C. until use.

### Expression Experiment Using Recombinant Viruses

HeLa cells were seeded in 6-well plate at 1.5E+06 cells/well/2 mL of complete medium (DMEM 4 Gibco ref. 41966-029; Glutamine 2 mM; Gentamicin 40 μg/mL; 10% FBS) the day prior infection. Cells were infected at MOI 0.1 with one of the following viruses COPTG19968, COPTG19967, COPTG19969 or VVTG18058. After 30 min of incubation the culture medium was discarded and replaced by 2 mL of DMEM; Glutamine 2 mM; Gentamicin 40 μg/mL. Cells were incubated 48 hours at 37° C. with 5% CO₂ and then the culture supernatants were recovered and treated as described above.

### CD40L and 4-1BBL Immunoblots

Twenty-five μL of samples (clarified supernatants) from infections/transfections were treated with Laemmli buffer (Biorad, 161-0747) containing (Reducing) or not (Non reducing) beta-mercaptoethanol. In case of reducing conditions, samples were heated at 95° C. for 3 minutes. Samples were then loaded on poly-acrylamide gel (TGX 4-15% Stain Free Biorad) and migration was performed in Tris Glycine SDS buffer (Biorad 161-0772). Western blot was performed using Transblot Turbo System (Biorad), set up on Midi Program High Molecular weight. Blots were then incubated with anti-FLAG-HRP conjugated antibody (Sigma A8592) at 2 μg/mL using Ibind Flex Western System (Invitrogen ref SLF2000). Positive controls were culture medium from an infection/transfection with an irrelevant plasmid (pTG19274) encoding a FLAG-tagged recombinant protein. Blots were incubated with HRP substrate (Amersham ECL Prime western blotting detection) and luminescence recorded by Chemidoc apparatus.

### CD40 ELISA

CD40-Fc was coated on Medisorp (Nunc) 96-well ELISA plate at 0.5 pg/mL in 50 mM carbonate buffer pH 9.6. Clarified supernatants of the infection/transfection experiment were diluted 10-fold added to the first well of the ELISA plate and further two-fold serially diluted in ELISA saturation buffer directly on the plate. The bound CD40L was detected by adding a non-competitive anti-human CD40L (MCA1561 Biorad) diluted 1000-fold in saturation buffer. Anti-Mouse Immunoglobulins-HRP conjugated antibody (Dako P0447) diluted 2000-fold was then added to each well. Finally, HRP substrate (3,3′,5,5′ tetramethylbenzidine) TMB was added to each well, absorbance 450 nm measured using TECAN microplate reader, and optical density (OD) 450 nm versus 1/dilution of culture supernatants were plotted using GraphPad prism software.

### CD40 Agonist Activity

HEK-Blue CD40L cells (Invivogen: hkb-cd40) are recombinant cells transformed to express both the human CD40 and a reporter enzyme (secreted embryonic alkaline phosphatase: SEAP) under the transcriptional control of a CD40 inducible promoter. Upon activation of CD40, SEAP is produced, and its enzymatic activity measured in culture medium following provider recommendation. The SEAP activity is proportional to the CD40 agonist activity.

The measures were performed following provider's instructions. Briefly, 50,000 HEK-Blue CD40L cells in 90 μL were distributed in 96-well plate and incubated with 20 μL of serial dilutions of clarified supernatants generated by the infection/transfection described above. After 24 hours of incubation at 37° C. and 5% CO₂, 40 μL of the culture medium were transferred with 160 μL of SEAP substrate (Invivogen: hb-det2) and incubated 3 hours at 37° C. Absorbance at 620 nm is measured using microplate reader and optical density versus 1/supernatant dilution plotted using GraphPad prism software.

### 4-1BB Agonist Activity

4-1BB Bioassay Promega kit (JA2351) was used according to providers instructions. Briefly, 25 μL of effector cells/well were mixed with 25 μL medium. Then 25 μL of serial dilutions of clarified supernatants were added to each well. Cells were then incubated at 37° C., 5% CO₂ for 6 h. 75 μL/well of reconstituted Bio-Glo (Promega, G7941) were added, and luminescence recorded using Berthold reader and MikroWin 2000 software. Luminescence versus 1/supernatant dilution was plotted using GraphPad prism software and four-parameter logistic curve analysis was performed.

### Vectorization and Virus Production

Recombinant viruses were generated using the same plasmids used in infection/transfection experiment described above. Briefly, chicken embryo fibroblasts (CEF) were infected with parental virus encoding GFP at the J2R (TK) locus and deleted of the I4L (RR) gene. Infected cells were transfected with the transfer plasmid carrying the expression cassette flanked by recombination harms (DNA sequences homolog of upstream and downstream J2R (TK) locus). Recombinant viruses are selected under binocular by picking “white” (i.e. GFP negative) lysis plaque. Expression cassette was checked by PCR amplification followed by DNA sequencing.

For in vivo experiments, recombinant viruses were produced on CEF (MOI 0.05, 72 hours) cultivated on F500. The cellular suspension containing the virus was homogenized by using a homogenizing mixer equipped with an in-line chamber. Large cellular debris were then eliminated by depth filtration using 5 μm pore size filters. The clarified viral suspension was subsequently concentrated and diafiltered with the formulation buffer (Saccharose 50 g/L, NaCl 50 mM, Tris 10 mM, Sodium Glutamate 10 mM, pH 8) using tangential flow filtration and 0.2 μm pore size hollow fiber microfiltration filters. Purified viruses were aliquoted and stored at −80° C. until use.

### EXAMPLES/RESULTS

### Expression of New Constructs

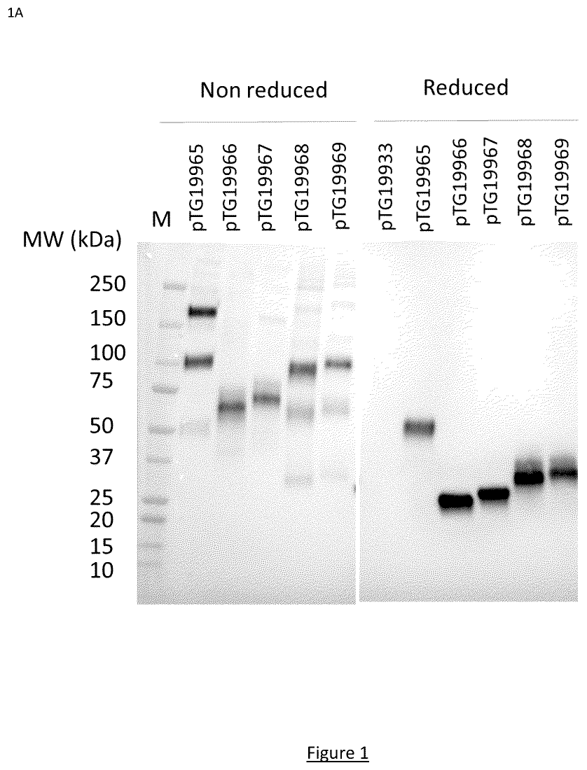

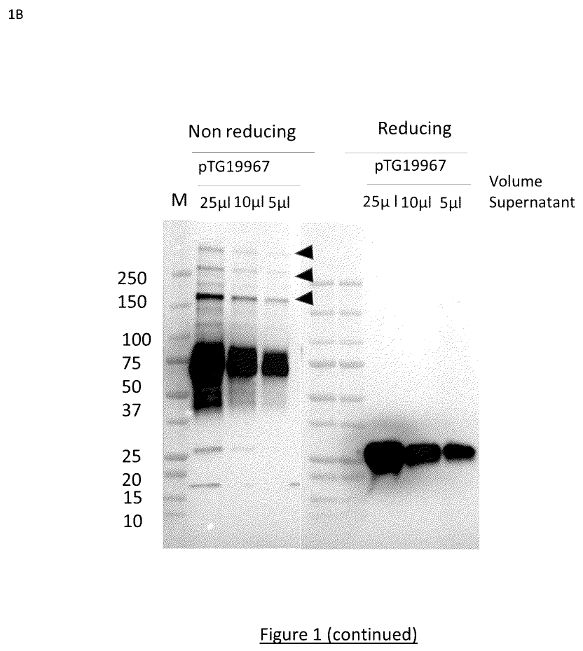

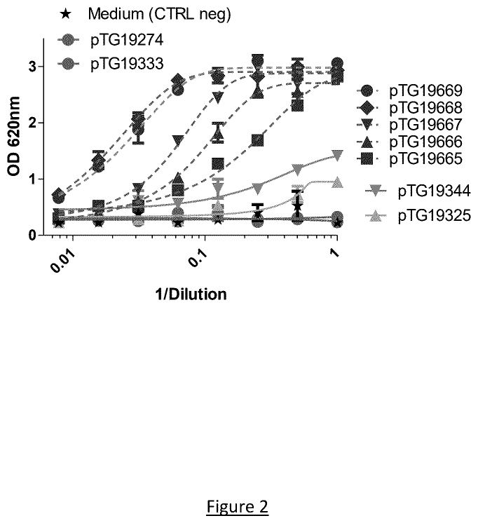

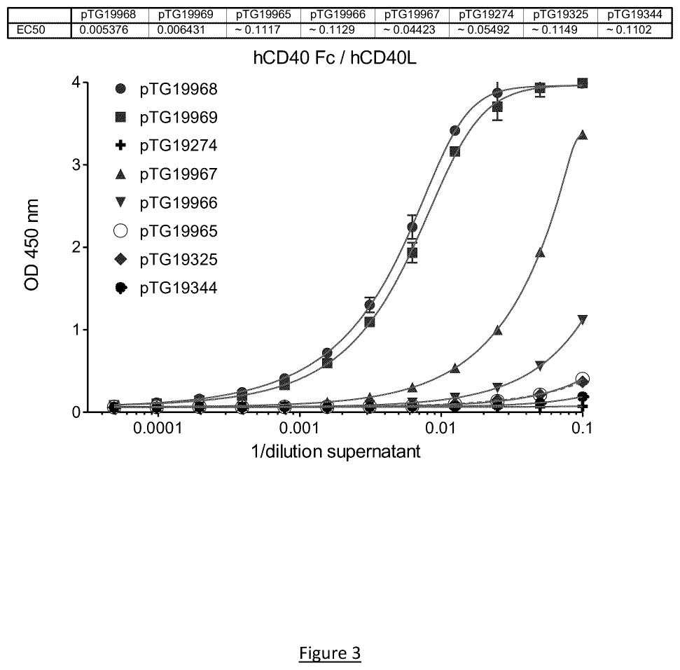

HeLa cells were infected and transfected with the transfer plasmids carrying the different SPD-TNFSF constructions as described above (pTG19965, pTG19966, pTG19967, pTG19968, pTG19969). Expression of recombinant SPD-TNFSF proteins in the culture supernatants were assessed by immunoblot using anti-FLAG tag for detection. FIGS. 1A and 1B show that all SPD-TNFSF fusion proteins were expressed at the expected monomer size in reducing condition and showed different degree of oligomerization in non-reducing conditions. Note that oligomers not locked by disulfide bonds are migrating as monomer in the denaturing condition of the electrophoresis.

It is also a particular benefit that the fusion protein according to the invention is of a smaller molecular size compared to existing fusion protein.

TABLE 3
Molecular size of SPD-TNFSF constructs
according to the invention.

| | SEQ ID NO of | | |
|---|---|---|---|
| Plasmid | constructs encoded | MW | Number of |
| reference | by the plasmid | (kDa) | residues |
| number encoding | | | |
| the construct | | | |
| pTG19325 | 50 | 46942 | 452 |
| pTG19965 | 52 | 37765 | 375 |
| pTG19966 | 2 | 21610 | 198 |
| pTG19967 | 10 | 22385 | 210 |
| pTG19968 | 3 | 24767 | 234 |
| pTG19969 | 11 | 25542 | 246 |

Interestingly, the construct encoded by pTG19967 also displayed a clear oligomerization on non-reducing condition with the trimer being the major band (at ^{˜}75 kDa) but hexamer (^{˜}150 kDa) and higher oligomers were also clearly visible.

It is also a particular interest that the fusion protein can form multimeric forms, without being restricted to trimeric forms in comparison to disclosures of the prior art.

### CD40 Agonist Activity of the Different SPD-TNFSF Constructions

The CD40 agonist activity of each construction was evaluated on HEK blue CD40L cells and compared to the proteins encoded by pTG19344 (soluble form CD40L extracellular domain) and pTG19325. A slight improvement of construct encoded by pTG19965 versus the constructs encoded by pTG19325 and pTG19344 was observed indicating that the change of peptide signal and/or the shortening of CD40L domain had an effect on the expression level and/or the agonist activity of the molecule. This benefit was highly amplified by the complete deletion of collagen domain with (construct encoded by pTG19967) or without (construct encoded to pTG19966) addition of a (GGGS)×3 linker between coiled-coil neck domain and CD40L. The downsizing of the collagen domain from 59 (GXX) repeats of the SPD full-length version (i.e. construct encoded by pTG19965) to 12 (GXX) repeats (i.e. constructs encoded by pTG19968, pTG19969) demonstrated the best CD40 agonist activity over all the other constructs. This result indicates that there is an optimal size of collagen domain to get the best CD40 agonist effect of the fusion. The collagen moiety of constructs encoded by pTG19968 and pTG19969 contain a N-Glycosylation site that might has some impact on the oligomer assembly and/or structure and hence on the CD40 agonist activity of the recombinant molecule. The present results indicate no significant effect of the N-glycosylation.

### Binding to CD40

An ELISA assay with immobilized recombinant human CD40 was set up to investigate the CD40 binding ability of all SPD-TNFSF constructs. In this assay a non-competitive anti-CD40L was used to detect the formation of CD40L/CD40 complex on the ELISA plate. In this assay, the clarified supernatants generated from constructs encoded by pTG19965 and pTG19325 infection/transfections had the same slight CD40 binding activity at the 1/10 dilution. The constructs with the modified SPD collagen domain demonstrated a clear CD40 improvement with the same ranking as the one observed in the CD40 agonist assay (i.e. constructs encoded by pTG19968>pTG19969>pTG19967>pTG19966). These results indicate that the better CD40 agonist activity observed of the collagen construct maybe due, at least in part, to a better binding to CD40.

### Effect of Collagen Domain

According to the invention, other number of (GXX) repeats around the 12 (GXX) repeats tested has been assessed. Therefore, different lengths or nature of the collagen (GXX) repeats were tested by i) shortening the original 12 (GXX) repeats to two constructions containing each 6 (GXX) repeats (with (construct encoded by pTG20038) and without (construct encoded by pTG20038) a N-glycosylation site) ii) selecting another 12 (GXX) repeats devoid of N-Glycosylation in another location into the SPD collagen domain (construct encoded by pTG20041), and iii) increasing the number of the (GXX) repeats to 19 (construct encoded by pTG20040) and 30 (construct encoded by pTG20042).

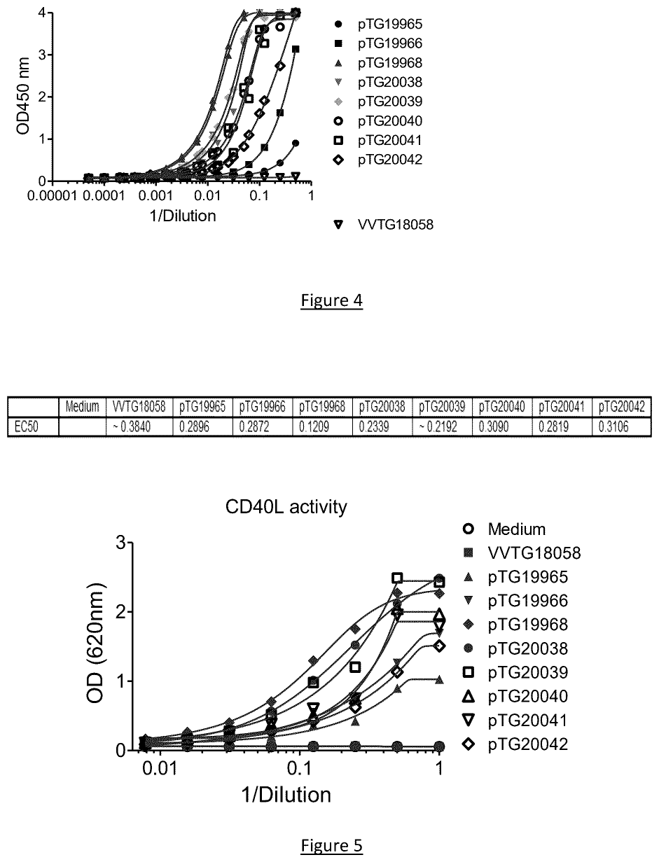

All those constructs have been expressed by the infection/transfection method described above and in parallel of the constructs encoded by pTG19968, pTG19966 and pTG19965 as references. The culture medium obtained were tested for CD40 binding by ELISA (FIG. 4) and on CD40 agonist assay (FIG. 5).

Both CD40 binding by ELISA (FIG. 4) and CD40 agonist assay (FIG. 5) demonstrate that the constructs were at best equivalent to the construct encoded by pTG19968. The two constructions with the 6 (GXX) repeats, with or without a N-glycosylation site, were slightly less potent for the biological activity than the construct encoded by pTG19968. The two constructions with the 12 (GXX) repeats (different from those of construct encoded by pTG19968) and 19 (GXX) repeats were clearly less potent for the biological activity than the construct encoded by pTG19968. Finally, the construction with 30 (GXX) repeats was the less potent for the biological activity of the constructs and equivalent to the construct encoded by pTG19966 for the biological activity.

Together these results demonstrate that the construct encoded by pTG19968 is the best construct among the ones tested although some alternative constructs (e.g. the constructs encoded by pTG19969, pTG19967 pTG20038, pTG20039) can be considered as almost equivalent.

Vectorization of pTG19968, pTG19969 and pTG19967

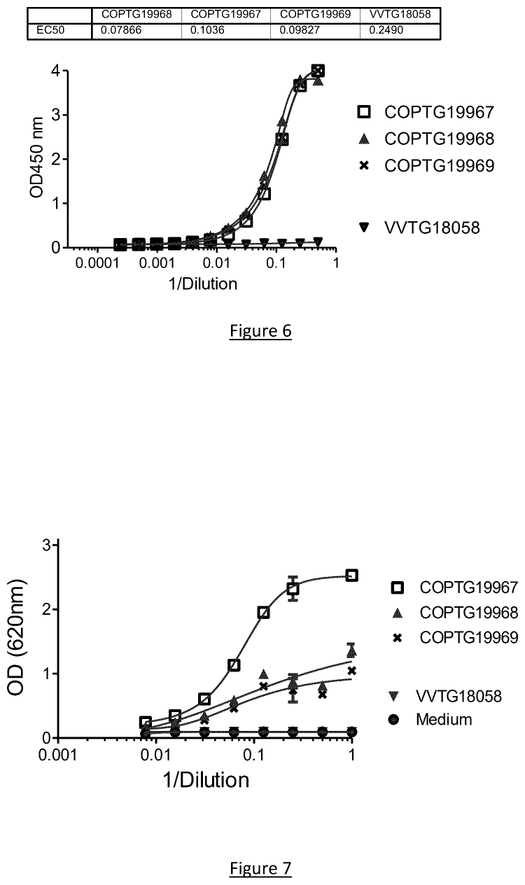

A selection of constructions (i.e. constructs encoded by pTG19968, pTG19969, and pTG19967) was inserted in vaccinia virus genome at the J2R (TK) locus by homologous recombination. The recombinant viruses were used to infect HeLa cells and the culture medium obtained after 48 hours of infection were tested for CD40 binding by ELISA (FIG. 6) and on CD40 agonist assay (FIG. 7). The EC50 for CD40 binding was increased (i.e. lower binding) by roughly ten-fold (FIG. 5, FIG. 7) for COPTG19968, and COPTG19969 compared to their infection/transfection counterparts.

Note that the curves shape did not even allow to calculate an accurate EC50 for COPTG19968 and COPTG19969. However, COPTG19967 yielded some CD40 agonist activity much better than those of COPTG19968 and COPTG19969. Among the SPD-TNFSF constructs vectorized in vaccinia virus, the one encoded by COPTG19967 is yielding the best CD40 agonist activity.

### 4-1-BBL Expression and 4-1-BB Agonist Activity of the SPD-TNFSF Construction

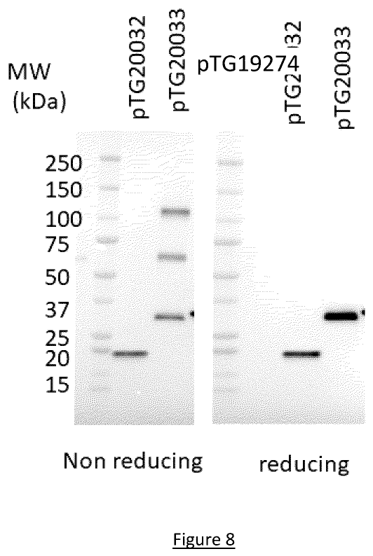

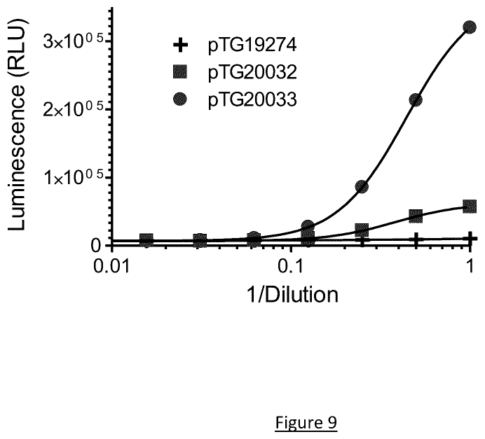

The potential of SPD-TNFSF fusion protein was extended to 4-1BBL as another member of the TNFSF. The CD40L ectodomain was replaced by the one of 4-1BBL in the construct encoded by pTG19968 as the fusion with the best agonist activity in the infection/transfection setting to generate construct encoded by pTG20033. 4-1BBL ectodomain alone was also cloned into the same backbone plasmid to generate the construct pTG20032 used further as reference. The two 4-1BBL molecules: 4-1BBL ectodomain alone encoded by pTG20032 or fused to the modified SPD with 12(GXX) repeats (construct encoded by pTG20033) were expressed at the same level by the infected/transfected cells (FIG. 8). Moreover, the pattern of oligomerization observed on non-reducing conditions was similar for both modified SPD with 12(GXX) repeats constructs (i.e. CD40L and 4-1BBL fusions). The 4-1BB agonist activity of the modified SPD with 12(GXX) repeats-4-1BBL construct was clearly superior to 4-1BBL alone (FIG. 9) although the gain of activity was less than the one observed for the equivalent CD40L construct.

### BIBLIOGRAPHIC REFERENCES

Adra et al., 1987, Gene 60: 65-74
Alfonso et al., 2002, 74(8):3815-383
Andtbacka et al., 2013, J. Clin. Oncol. 31, abstract number LBA9008
Antoine et al., 1998, Virol, 244: 365-396
Ara et al., 2018, ImmunoTargets and Therapy, 7, 55-61
Beatty et al., 2017, Expert Rev Anticancer Ther.; 17(2):175-186
Boviatsis et al., Gene Ther. 1: 323-31
Brandler and Tangy, 2008, CIMID 31: 271
Bremer et al., 2013, ISRN Oncol.; 2013:371854
Broyles et al., 1993, Virol. 195: 863-5
Caroll et al., 1997, Virology 238: 198-211
Chakrabarti et al., 1997, Biotechniques 23: 1094-7
Chambers et al., 1995, Proc. Natl. Acad. Sci. USA 92: 1411-5
Chan, 2008, Eur. J. Immunol. 38, 2964-2968
Chartier et al., 1996, J. Virol. 70: 4805-10
Chaurasiya et al, 2020, Cancer Gene Ther 27, 125-135
Choi et al, 2018, Surgery Volume 163, Issue 2, 336-342
Chu et al., 1997, J. Exp. Med. 186: 1623
Colberre-Garapin et al., 1981, J. Mol. Biol. 150: 1
Croft M et al., 2017, Nat Rev Rheumatol.; 13(4):217-233
Erbs et al., 2008, Cancer Gene Ther. 15(1):18-28
Fallaux et al., 1998, Human Gene Ther. 9: 1909-17
Foloppe et al., 2008, Gene Ther., 15: 1361-71
Freeman et al., 2006, Mol. Ther. 13(1):221-8
Friedman-Kien et al., 1963, Science 140: 1335-6
Geevarghese et al., 2010, Hum. Gene Ther. 21(9):1119-28
Gupta et al., 2013, Immunol Res., 57(1-3):303-10
Guse et al., 2011, Expert Opinion Biol. Ther. 11(5):595-608
Hammond et al, 1997, J. Virol Methods 66: 135-8
Haswell et al. 2001, Eur J Immunol., 31(10):3094-100
Hautaniemi et al., 2010, J. Gen. Virol. 91: 1560-76
J. R. Robinson in “Sustained and Controlled Release Drug Delivery Systems”, ed., Marcel Dekker, Inc., New York, 1978
Kaufmann et al., 2013, J. Invest. Dermatol. 133(4):1034-42
Khuri et al., 2000, Nat. Med 6(8):879-85
Kirn et al., 2001, Nat. Med. 7: 781
Kritsch et al., 2005, J. Chromatogr Anal. Technol. Biomed. Life Sci. 822: 263-70
Kucka et al., 2020, Front Cell Dev Biol., 8:615141
Kumar et al., 1990, Virology 179: 151-8
Lauterbach et al., 2013, Front Immunol., 27; 4:251
Li et al. MAbs. 2016; 8(1):113-9
Lorence et al., 2007, Curr. Cancer Drug Targets 7(2):157-67
Martuza et al., 1991, Science 252: 854-6
Mayr et al., 1975, Infection 3: 6-14
McDonald et al., 2006; Breast Cancer Treat. 99(2):177-84
Mineta et al., 1994, Cancer Res. 54: 3363-66
Mulligan and Berg, 1981, Proc. Natl. Acad. Sci. USA 78: 2072
O'Hare et al., 1981, Proc. Natl. Acad. Sci. USA 78: 1527
Paul et al., 2002, Cancer gene Ther. 9: 470-7
Pesonen et al., 2012, Cancer Res., 72(7)
Phuangsab et al., 2001, Cancer Lett. 172(1):27-36
Pyles et al., 1994, J. Virol. 68: 4963-72
Ribi et al., 1986, Immunology and Immunopharmacology of Bacterial Endotoxins, Plenum Publ. Corp., NY, p407-419
Richards et al., 2020, Hum Vaccin Immunother. 16(2):377-387
Rudin et al., 2011, Clin. Cancer. Res. 17(4):888-95
Sambrook et al., 2001, Molecular Cloning-A Laboratory Manuel, Cold Spring Harbor Laboratory
Santerre et al., 1984, Gene 30: 147
Scott-Algara et al., 2010 PLOS One 5(1), e8761
Skeate et al., 2020, Front Immunol., 15; 11:922
Smorlesi, 2005, Gene Ther. 12: 1324
Stojdl et al., 2000, Nat. Med. 6(7):821-5
Stojdl et al., 2003, Cancer Cell 4(4):263-75
Suader, 2000, J. Am Acad Dermatol. 43:S6
Sumino et al., 1998, J. Virol. 72: 4931
Sutter and Moss, 1992, Proc. Natl. Acad. Sci. USA 89: 10847-51
Tartaglia et al., 1992, Virol. 188(I):217-32
Tikkanen et al., 2004, J. Gen. Virol. 85: 1413-8
Tritel et al., 2003, J. Immunol. 171: 2358
Vinay et al., 2009, Cell Biol Int.; 33(4):453-465
Vonderheide et al., 2007, J Clin Oncol., 25(7):876-883
Wigler et al., 1980, Proc. Natl. Acad. Sci. USA 77: 3567
Wong et al., 2010, Viruses 2: 78-106
Xia et al., 2004, Ai Zheng 23(12):1666-70
Zang et al., 2007, Cancer Res. 67:10038-46
Zhou et al., 2006, Blood 107, 2461-2469
U.S. Pat. No. 6,686,152
U.S. Pat. No. 6,984,720
U.S. Pat. No. 7,109,003
U.S. Pat. No. 7,291,331
U.S. Pat. No. 8,017,114
U.S. Pat. No. 8,143,379
U.S. Pat. No. 8,491,895
U.S. Pat. No. 5,168,062
U.S. Pat. No. 5,494,807
U.S. Pat. No. 6,686,152
WO01/42298
WO01/49866
WO01/66137
WO03/053463
WO03/106498
WO2000/037504
WO2004/004771
WO2004/056875
WO2005/035584
WO2005/042728
WO2006/108846
WO2006/121168
WO2007/056847
WO2007/113648
WO2007/123737
WO2007/147528
WO2007/147529
WO2008/114021
WO2008/129058
WO2008/138533
WO2008/138649
WO2008/156712
WO2009/007120
WO2009/014708
WO2009/065546
WO2009/065547
WO2009/073569
WO2009/100521
WO2009/114335
WO2010/106051
WO2010/130753
WO2010/130756
WO2012/001075
WO2012/004367
WO2012/122444
WO2013/022764
WO2013/043569
WO2014/047350
WO2014/053571
WO2016/087457
WO2016/196237
WO2017/068186
WO2017/184619
WO2018/071500
WO2018/187613
WO2019/023347
WO2019/158645
WO2020/019135
WO2020/093023
WO97/20574
WO98/02522
WO98/10088
WO98/56415

1. A SPD-TNFSF fusion protein comprising:
a N-terminus domain
a coiled-coil neck domain of surfactant protein-D (SPD) between the N-terminus domain and the C-terminus position, and
a TNF-superfamily (TNFSF) ligand, or a receptor binding domain thereof in C-terminus position.

2. A SPD-TNFSF fusion protein according to claim 1, wherein said fusion protein further comprises a collagen domain between the N-terminus domain and the coiled-coil neck domain of SPD.

3. A SPD-TNFSF fusion protein according to claim 2, wherein said collagen domain comprises between 1 and 40 (GXX) repeats, preferably between 3 and 30 (GXX) repeats, preferably between 6 and 20 (GXX) repeats, more preferably 12 (GXX) repeats, wherein X is an amino acid, and G is a glycine amino acid.

4. A SPD-TNFSF fusion protein according to anyone of claims 1 to 3, wherein said fusion protein further comprises a linker between the coiled-coil neck domain and the TNF-superfamily ligand or the receptor binding domain thereof.

5. A SPD-TNFSF fusion protein according to claim 4, wherein said linker is a glycine/serine linker and has a length of 4-20 amino acids, preferably 8-16, more preferably 12 amino acids.

6. A SPD-TNFSF fusion protein according to anyone of claims 1 to 5, wherein said TNF-superfamily ligand is selected from CD40L, 4-1-BBL, CD70, OX40L, TNF, GITRL, LIGHT, FASL, TWEAK, APRIL, RANKL, TRAIL, CD30L, NGF, Baff, LTβ, LTα, LTαβ2, TL1A, TLA, EDA more preferably CD40L or 4-1-BBL.

7. A SPD-TNFSF fusion protein according to anyone of claims 1 to 6, wherein N-terminus domain having at least 85%, preferably at least 90%, and more preferably at least 95% identity with the amino acid sequence shown in SEQ ID NO:1

8. A SPD-TNFSF fusion protein according to anyone of claims 1 to 7, comprising or consisting of a sequence selected from SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11.

9. A trimeric fusion protein comprising three fusion proteins according to claims 1 to 8.

10. A multimeric fusion protein comprises a plurality of trimeric fusion proteins according to claim 9 which forms a hexamer, a dodecamer, an octadecamer or a highly-order oligomer, preferably an hexamer, and more preferably a dodecamer.

11. An isolated nucleotide sequence encoding a fusion protein as defined in anyone of claims 1 to 8.

12. An isolated nucleotide sequence according to claim 11, wherein said sequence comprising or consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21.

13. A mRNA comprising a nucleotide sequence as defined in claim 11.

14. A plasmid comprising a nucleotide sequence as defined in claim 11 or 12.

15. A virus comprising a nucleotide sequence as defined in claim 11 or 12.

16. A virus according to claim 15, wherein said virus is an oncolytic or a non-oncolytic virus.

17. A virus according to claim 16, wherein said oncolytic virus is selected from the group consisting of poxvirus, herpes virus, reovirus, Seneca Valley virus (SVV), vesicular stomatitis virus (VSV), Newcastle disease virus (NDV), morbillivirus, retrovirus, adenovirus, adenovirus-associated virus (AAV), herpes simplex virus (HSV), measles virus, foamy virus, alpha virus, lentivirus, influenza virus, Sindbis virus, rhabdovirus, picornavirus, coxsackievirus, parvovirus or chimeras thereof.

18. A virus according to claim 17, wherein said oncolytic virus is a poxvirus and belongs to the Orthopoxvirus genus preferably selected from the group consisting of Vaccinia virus, cowpox virus, canarypox virus and ectromelia virus.

19. A virus according to claim 18, wherein said oncolytic poxvirus is a vaccinia virus and in particular a vaccinia virus selected from the group of Elstree, Wyeth, Copenhagen, Lister, Tian Tian and Western Reserve strains.

20. A virus according to claim 17, wherein said oncolytic virus is a poxvirus and belongs to the leporipoxvirus genus, preferably selected from the group consisting of myxoma virus, rabbit fibroma virus and squirrel fibroma virus, preferably myxoma virus.

21. A virus according to anyone of claims 18 to 20, wherein said oncolytic poxvirus is a virus defective for thymidine kinase (TK) activity resulting from inactivating mutations in the J2R viral gene.

22. A virus according to claim 21, wherein said oncolytic poxvirus is a virus defective for ribonucleotide reductase (RR) activity resulting from inactivating mutations in the viral I4L and/or F4L gene(s).

23. A virus according to anyone of claim 21 or 22, wherein said oncolytic poxvirus is a virus defective for m2 functions resulting from inactivating mutations in the M2L viral gene.

24. A virus according to claim 16, wherein said non-oncolytic virus is a poxvirus.

25. A virus according to claim 24 wherein said poxvirus is selected from the group consisting of Pseudocowpox virus (PCPV), Modified vaccinia Virus Ankara (MVA), highly attenuated vaccinia virus strain (NYVAC), Swinepox virus (SWPV), Fowlpox virus (FPV) or chimeras thereof

26. A method for producing the virus of any one of claims 15 to 25 comprising the steps of a) preparing a producer cell b) transfecting or infecting the prepared producer cell with the virus, c) culturing the transfected or infected producer cell under suitable conditions so as to allow the production of the virus, d) recovering the produced virus from the culture of said producer cell and optionally e) purifying said recovered virus.

27. A cell comprising a nucleotide sequence as defined in claim 11 or 12, or a mRNA as defined in claim 13, or a plasmid as defined in claim 14, or a virus as defined according to anyone of claims 15 to 25.

28. A SPD-TNFSF fusion protein as defined in anyone of claims 1 to 8, a trimeric fusion protein according to claim 9, a multimeric fusion protein according to claim 10, a nucleotide sequence according to claim 11 or 12, a mRNA according to claim 13, a plasmid according to claim 14, a virus according to anyone of claims 15 to 25, or a cell according to claim 27 for use in medicine.

29. A SPD-TNFSF fusion protein as defined in anyone of claims 1 to 8, a trimeric fusion protein according to claim 9, a multimeric fusion protein according to claim 10, a nucleotide sequence according to claim 11 or 12, a mRNA according to claim 13, a plasmid according to claim 14, a virus according to anyone of claims 15 to 25, or a cell according to claim 27 for use in the treatment of proliferative diseases such as cancer and disorders associated with dysfunction of TNF cytokines such as infectious diseases, inflammatory diseases, metabolic diseases, autoimmune diseases, degenerative diseases, apoptosis-associated diseases and transplant rejections.

30. A SPD-TNFSF fusion protein as defined in anyone of claims 1 to 8, a trimeric fusion protein according to claim 9, a multimeric fusion protein according to claim 10, a nucleotide sequence according to claim 11 or 12, a mRNA according to claim 13, a plasmid according to claim 14, a virus according to anyone of claims 15 to 25, or a cell according to claim 27 for use in the treatment of cancer.

31. A SPD-TNFSF fusion protein as defined in anyone of claims 1 to 8, a trimeric fusion protein according to claim 9, a multimeric fusion protein according to claim 10, a nucleotide sequence according to claim 11 or 12, a mRNA according to claim 13, a plasmid according to claim 14, a virus according to anyone of claims 15 to 25, or a cell according to claim 27 in combination with one or more chemotherapeutic drugs or immunotherapeutic products effective for use in the treatment of cancer.

32. A pharmaceutical composition comprising or consisting of a SPD-TNFSF fusion protein as defined in anyone of claims 1 to 8, a trimeric fusion protein according to claim 9, a multimeric fusion protein according to claim 10, a nucleotide sequence according to claim 11 or 12, a mRNA according to claim 13, a plasmid according to claim 14, a virus according to anyone of claims 15 to 25, or a cell according to claim 27 and optionally a pharmaceutically acceptable diluent, carrier, vehicle and/or excipient.

33. A pharmaceutical composition according to claim 32, wherein said pharmaceutical composition further comprises one or more effective chemotherapeutic drugs or immunotherapeutic products.

34. A pharmaceutical composition according to claim 32 or 33 for use in the treatment of cancer.

35. A pharmaceutical composition for use according to anyone of claims 32 to 34, wherein said pharmaceutical composition is administered via parenteral route, more preferably via intravenous, subcutaneous, or intramuscular route, and even more preferably via intravenous route.

36. A method of treatment of cancer in a subject comprising administering to the subject a therapeutically effective amount of a fusion protein as defined in anyone of claims 1 to 8, a trimeric fusion protein according to claim 9, a multimeric fusion protein according to claim 10, a nucleotide sequence according to claim 11 or 12, a mRNA according to claim 13, a plasmid according to claim 14, a virus according to anyone of claims 15 to 25, or a cell according to claim 27, or a pharmaceutical composition according to anyone of claims 32 to 34.
